(19) United States
(12) Patent Application Publication
(10) Pub. No.: US 2024/0066521 A1
(43) Pub. Date: Feb. 29, 2024

(54) MACHINE READABLE DIAGNOSTIC TEST DEVICES AND METHODS AND APPARATUS TO MAKE AND/OR PROCESS THE SAME

(71) Applicant: Abbott Rapid Diagnostics International Unlimited Company, Dublin 2 (IE)

(72) Inventors: Thomas Uhlig, Jena (DE); Thomas Ullrich, Jena (DE); Stefan Kügler, Jena (DE)

(21) Appl. No.: 18/456,330

(22) Filed: Aug. 25, 2023

Related U.S. Application Data
(60) Provisional application No. 63/401,073, filed on Aug. 25, 2022.
(60) Provisional application No. 63/432,924, filed on Dec. 15, 2022.
(60) Provisional application No. 63/401,075, filed on Aug. 25, 2022.

(51) Int. Cl. B01L 3/00

(52) U.S. Cl. CPC B01L 3/5023; B01L 2300/0645; B01L 2300/123

(57) ABSTRACT

Methods, apparatus, systems, and articles of manufacture to make and/or process a diagnostic test device are disclosed. An example apparatus includes a first portion including an opening; a second portion coupled to the first portion and house a lateral flow assay strip, the second portion including a first clip; and a push button located within the opening of the first portion, the push button moveable from a first position to a second position, the push button including a second clip to engage the first clip of the second portion to maintain the push button in the second position when moved into the second position.

### RELATED APPLICATIONS

This patent claims the benefit of U.S. Provisional Patent Application No. 63/401,073, which was filed on Aug. 25, 2022, U.S. Provisional Patent Application No. 63/401,075, which was filed on Aug. 25, 2022, and U.S. Provisional Patent Application No. 63/432,924, which was filed on Dec. 15, 2022. U.S. Provisional Patent Application No. 63/401,073, U.S. Provisional Patent Application No. 63/401,075, and U.S. Provisional Patent Application No. 63/432,924 are hereby incorporated herein by reference in their entireties. Priority to U.S. Provisional Patent Application No. 63/401,073, U.S. Provisional Patent Application No. 63/401,075, and U.S. Provisional Patent Application No. 63/432,924 is hereby claimed. Additionally, U.S. patent application Ser. No. 17/928,188 and U.S. patent application Ser. No. 17/927,648 are hereby incorporated herein by reference in their entries.

### FIELD OF THE DISCLOSURE

This disclosure relates generally to biosensors, and, more particularly, to a machine readable diagnostic test devices and methods and apparatus to make and/or process the same.

### BACKGROUND

A biosensor (e.g., a lateral flow device, such as a lateral flow assay (LFA)) is a device that is capable of detecting a condition, disease, analyte, etc., in a human or animal based on a sample (e.g., a blood sample, a saliva sample, a urine sample, etc.) from the human or animal. LFAs have been used to detect the presence of a target analyte to determine pregnancy, presence of HIV, presence of Covid-19, presence of Ebola, presence of different toxins, etc.

### BRIEF DESCRIPTION OF THE DRAWINGS

FIG. 1A is an example environment including an example machine readable lateral flow immunoassay generator to generate a machine readable lateral flow immunoassay described in conjunction with examples disclosed herein.

FIG. 1B is a side view of the example machine readable lateral flow immunoassay of FIG. 1A.

FIG. 2A illustrates a top view of an example implementation of the machine readable lateral flow immunoassay of FIG. 1A.

FIG. 2B illustrates a side view of the machine readable lateral flow immunoassay of FIG. 2A.

FIG. 2C illustrates example conjugates that may attach to an immobilized antigen and/or antibody on a test line of the machine readable lateral flow immunoassay of FIGS. 1A-2B.

FIGS. 2D-2F illustrate example implementations of front end channels that may be used in the machine readable lateral flow immunoassay of FIGS. 1-2D.

FIG. 2G illustrates an example housing for the example LFA device of FIG. 1A in a first position.

FIG. 2H illustrates the example housing for the LFA device of FIG. 1A in a second position.

FIG. 2I illustrates an exploded view of the example housing of FIGS. 2G and 2H.

FIG. 2J illustrates an over-the-top view of a cross section of the housing along the A-A line of FIG. 2G and rotated.

FIG. 2K illustrates an example housing for the example LFA device of FIG. 1A in a first position.

FIG. 2L illustrates the example housing for the LFA device of FIG. 1A in a second position.

FIG. 2M illustrates an exploded view of the example housing of FIGS. 2K and 2L.

FIG. 2N illustrates an over-the-top view of a cross section taken along the B-B line of FIG. 2K.

FIG. 2O illustrates two partial cross sectional views of the example housing of FIGS. 2K and 2L with the slider in the first position and the second position. The cross section of the top figure and the bottom figure in FIG. 2O is taken along the C-C line of FIG. 2K and rotated 180 degrees horizontally.

FIG. 2P illustrates operation of the lateral flow assay devices of FIGS. 1A-2D and/or 2K-2O in conjunction with a holding compartment.

FIG. 2Q illustrates an alternative operation of the lateral flow assay devices of FIGS. 1A-2D and/or 2K-2O in conjunction with a holding compartment.

FIG. 3 is a block diagram of an example implementation of the machine readable lateral flow immunoassay generator of FIG. 1A.

FIGS. 4-5 illustrate flowcharts representative of example machine readable instructions which may be executed to implement the machine readable lateral flow immunoassay generator of FIGS. 1A and/or 3.

FIG. 6 is a block diagram of an example processing platform structured to execute the instructions of FIGS. 4-5 to implement the lateral flow immunoassay generator of FIGS. 1A and/or 3.

FIG. 7 is a block diagram of an example software distribution platform to distribute software (e.g., software corresponding to the example computer readable instructions of FIGS. 3-4) to client devices such as consumers (e.g., for license, sale and/or use), retailers (e.g., for sale, re-sale, license, and/or sub-license), and/or original equipment manufacturers (OEMs) (e.g., for inclusion in products to be distributed to, for example, retailers and/or to direct buy customers).

The figures are not to scale. Instead, the thickness of the layers or regions may be enlarged in the drawings. In general, the same reference numbers will be used throughout the drawing(s) and accompanying written description to refer to the same or like parts. As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Stating that any part is in “contact” with another part means that there is no intermediate part between the two parts. Although the figures show layers and regions with clean lines and boundaries, some or all of these lines and/or boundaries may be idealized. In reality, the boundaries and/or lines may be unobservable, blended, and/or irregular.

### DETAILED DESCRIPTION

Rapid diagnostic tests include a biosensor or test strip device (e.g., lateral flow immunoassay (LFA)), which is a device including a first region to obtain a sample (e.g., blood, urine, saliva, nasal, etc.) and a second region that changes (e.g., changes color and/or experiences another change in a physical property) when a target analyte corresponding to a particular disease or condition is present in the sample. For example, a user applies the sample to a sample pad of a test strip device, or simply “test strip” (e.g., an LFA, etc.). Once applied, the sample migrates along the test strip to a conjugate pad that contains conjugates (e.g., detectable labels, tags, linkers, antibodies, antigens, etc.) specific to the target analyte. If the sample includes the target analyte, a reaction (e.g., a chemical reaction, biochemical reaction, physical reaction, etc.) will occur on the conjugate pad to bind the target analyte with the conjugates. The test strip also includes a test line that contains molecules (e.g., immobilized antigens, antibodies, analytes, aptamers, etc., specific to the target analyte), which bind the first set of conjugate molecules (e.g., probe molecules) from the conjugate pad. For example, if the analyte of interest is an antibody, the positive test area includes immobilized antigen. If the analyte of interest is an antigen, the positive test area includes immobilized antibody. The labeled substance or conjugate includes a first binding component that is able to bind the analyte of interest and, in some examples, a second visualization component. Accordingly, when the sample (e.g., including the bounded target analyte) flows to a test zone (e.g., a reaction zone), the antibodies, analytes, or antigens of the test line bind to the bounded target analyte, thereby immobilizing the target analyte. In some test strips, the immobilized target analytes result in a visual output that identifies that the target analyte was present in the sample. Accordingly, a scanner or user can identify whether the target analyte (e.g., corresponding to a condition or disease) is present in the sample based on the color of the test zone.

“Target analyte”, “analyte” or “analyte of interest” refers to the compound or the composition to be detected or measured from the sample, which has at least one epitope or binding site. The analyte can be any substance for which there exists a naturally occurring analyte-specific binding member or for which an analyte-specific binding member can be prepared. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), and/or metabolites of or antibodies to any of the above substances. The term “analyte” also includes any antigenic substances, haptens, antibodies, macromolecules, and/or combinations thereof.

“Label” refers to any substance which is capable of producing a signal that is detectable by visual and/or instrumental means. Various labels suitable for use in examples disclosed herein include labels that produce signals through chemical and/or physical means. Examples include enzymes and substrates, chromagens, fluorescent compounds, chemiluminescent compounds, colored or colorable organic polymer latex particles, liposomes, and/or other vesicles containing directly visible substances. In some examples, radioactive labels, colloidal metallic particles, and/or colloidal non-metallic particles are employed. In some examples, labels include colloidal gold and latex particles.

“Labeled substance” or “conjugate” refers to a substance that includes a detectable label attached to a specific binding member. The attachment may be covalent or non-covalent binding and may include nucleic acid hybridization. The label allows the labeled substance to produce a detectable signal that is directly or indirectly related to the amount of analyte in a test sample. The specific binding member component of the labeled substance is selected to bind directly or indirectly to the analyte.

“Specific binding member” refers to a member of a specific binding pair (e.g., two different molecules wherein one of the molecules specifically binds to the second molecule through chemical or physical means). If the specific binding member is an immunoreactant, it can be, for example, an antibody, analyte, antigen, hapten, or complex thereof, and if an antibody is used, it can be a monoclonal or polyclonal antibody, a recombinant protein or antibody, a chimeric antibody, a mixture(s), or fragment(s) thereof, as well as a mixture of an antibody and other specific binding members. Specific examples of specific binding members include biotin and avidin, an antibody and its corresponding antigen (both having no relation to a sample to be assayed), a single stranded nucleic acid and its complement, and the like.

A “test strip” or “LFA” can include one or more bibulous or non-bibulous materials. If a test strip includes more than one material, the one or more materials are preferably in fluid communication. One material of a test strip may be overlaid on another material of the test strip, such as for example, filter paper overlaid on nitrocellulose. Additionally or alternatively, a test strip may include a region including one or more materials (e.g., media) followed by a region including one or more different materials. In this case, the regions are in fluid communication and may or may not partially overlap one another. Suitable materials for test strips include, but are not limited to, materials derived from cellulose, such as filter paper, chromatographic paper, nitrocellulose, and cellulose acetate, as well as materials made of glass fibers, nylon, dacron, polyvinyl chloride (PVC), polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH or specific gravity of an applied urine sample, to ensure optimal test conditions.

The material or materials can be a single structure such as a sheet cut into strips, or it can be several strips or particulate material bound to a support or solid surface such as found, for example, in thin-layer chromatography and may have an absorbent pad either as an integral part or in liquid contact. The material can also be a sheet having lanes thereon, capable of spotting to induce lane formation, wherein a separate assay can be conducted in each lane. The material can have a rectangular, circular, oval, triagonal or other shape provided that there is at least one direction of traversal of a test solution by capillary migration. Other directions of traversal may occur such as in an oval or circular piece contacted in the center with the test solution. However, the main consideration is that there be at least one direction of flow to a predetermined site. In the following discussion test strips will be described by way of illustration and not limitation.

The support for the test strip, where a support is desired or necessary, will normally be water insoluble, frequently non-porous and rigid but may be elastic, usually hydrophobic, and porous and usually will be of the same length and width as the strip but may be larger or smaller. The support material can be transparent, and, when a test device disclosed herein is assembled, a transparent support material can be on the side of the test strip that can be viewed by the user, such that the transparent support material forms a protective layer over the test strip where it may be exposed to the external environment, such as by an aperture in the front of a test device. A wide variety of mobilizable and non-mobilizable materials, both natural and synthetic, and combinations thereof, may be employed provided only that the support does not interfere with the capillary action of the material or materials, or non-specifically bind assay components, or interfere with the signal producing system. Illustrative polymers include polyethylene, polypropylene, poly (4-methylbutene), polystyrene, polymethacrylate, poly (ethylene terephthalate), nylon, poly (vinyl butyrate), glass, ceramics, metals, and the like. Elastic supports may be made of polyurethane, neoprene, latex, silicone rubber and the like. Throughout this description, LFAs are described with the understanding that description of the LFAs applies to other types of test strips.

In some conventional LFAs, the test results appear as faint color changes that result in an increase in user error when reading. For example, although an LFA may output a color corresponding to a positive result, if the color is faint and/or the lighting conditions are poor, a user may interpret the test as negative. Additionally, because some conventional LFAs rely on visual cues to determine a result, the test lines that correspond to a result need to be sufficiently spaced and/or sufficiently limited to avoid confusion or reading errors. Examples disclosed herein generate an LFA that is machine readable to provide an objective and automated result generation through an algorithm that reduces and/or otherwise eliminates false positives and/or false negatives due to human error. Additionally, the use of an LFA that does not rely on visual cues allows for more test zones closer together without the risk of misreading visual results.

Examples disclosed herein provide an LFA that can be read using a smartphone application (e.g., directly or via a dedicated reader). Although some smartphone applications may be capable of reading the result of an LFA-based test, such smartphone applications may output inaccurate results due to poor lighting conditions, LFAs with weak visual indicators, and/or poor quality sensors on smartphones.

Examples disclosed herein generate an LFA to address conventional smartphone application errors and/or human errors that correspond to inaccurate results of the conventional LFA readers. Examples disclosed herein provide a signal corresponding to the test result (e.g., one or more analog current and/or voltage values corresponding to a test result, one or more digital current and/or voltage values corresponding to the test result, one or more logic values corresponding to a test result, etc.), which provide more accurate readings than analog systems. Examples disclosed herein correspond to a machine readable LFA device that does not rely on conventional visual indicators to determine the result of a fluid sample-based test (e.g., the LFA-based test). In examples disclosed herein, the LFA provides machine-readable results to a smartphone application. Thus, the results are objective and readable without human interpretation, and there is no operator subjectivity, which increases the accuracy of the results. There also is no need to include a read window or similar for a visual read of the results of the LFA device. As disclosed further herein, the example LFA device does not need to house a battery (e.g., a device that stores energy). For example, the LFA device is batteryless, not connected to power by wire, and is powered by the electromagnetic field generated by a user device such as a reader. Furthermore, the accurate results can be readily transmitted to an external database and/or external server such as, for example, a remote database and/or server located at or otherwise associated with an electronic medical record (EMR), a government agency, a non-government agency (NGO), a doctor's office, a hospital, a hospital information system, a laboratory information management software (LIMS) system, a stock consumption monitor, a clinic, and/or other medical facility, a medical device manufacturer, a medical organization, a health information system, and/or other external entity. In this manner, large scale test results can be generated, collected, and integrated into other healthcare systems digitally to eliminate human-based transcription errors. The examples disclosed herein also enable self-testing including, for example, testing by non-medical personnel because untrained self-testers do not need knowledge of how to interpret the results. Self-testing may be incorporated into over-the-counter or consumer devices. Examples disclosed herein may be incorporated into disposable point-of-care devices.

Examples disclosed herein include an LFA device that includes a wireless chip to obtain data corresponding to whether an analyte is present in a sample based on an electrical signal generated on the LFA device and to transmit the data to a reader. The determination of the test result is based on a comparison of the electrical signal to one or more thresholds. The comparison can be done on the wireless chip of the LFA and/or at an external reader. In some examples, the wireless chip obtains an analog current and/or voltage values from electrodes placed in contact with the porous membrane of the LFA device and transmits the analog current and/or voltage values to the reader via an antenna. In this manner, the reader can compare the analog values to one or more threshold to determine if a test result is positive or negative. For example, if the current and/or voltage value(s) is above a threshold, the reader determines the test corresponding to the current and/or voltage value(s) is positive. Likewise, if the current and/or voltage value(s) is below the threshold, the reader determines the test corresponding to the current and/or voltage value(s) is negative. In some examples, the wireless chip obtains analog current and/or voltage values, converts the analog values to digital values, and transmits the digital values to the reader. In this manner, the reader can compare the digital value(s) to one or more threshold to determine a test result. In some examples, the wireless chip obtains the analog voltage and/or current values and compares the values (e.g., with or without converting to digital values) to one or more threshold to generate a logic value (e.g., high or low) corresponding to a positive or negative test and transmits the test results to the reader.

Examples disclosed herein include multiple techniques for generating an electrical signal on the LFA device indicative of the presence or absence of the analyte of interest. For example, to obtain the one or more electrical signals that correspond to one or more test results, examples disclosed herein may utilize a bioelectrochemical mechanism (e.g., a device that generates energy) at the test and/or control lines of the LFA device. As further described below, a bioelectrochemical mechanism includes a bioelectrochemical cell (e.g., structuring the porous membrane to act as the physical cell) that, when a target analyte is present, generates an electrical signal at the porous membrane. The bioelectrochemical cell may be known as or otherwise include a potentiometric cell, a concentration-cell, a fuel cell, a biofuel cell, etc. In some examples, the bioelectrochemical cell generates the electrical signal due to one or more bioelectrochemical reactions that occur on the porous membrane. The wireless chip of the LFA device can measure the electrical signal by sensing a current and/or a voltage drop between electrodes of the bioelectrochemical cell corresponding to the porous membrane.

In some examples of the bioelectrochemical cell disclosed herein, a bioelectrochemical cell is attached to a test zone (e.g., test line, test region, etc.) and/or control zone (control line) of the porous media (e.g., a membrane, a paper, and/or other compartment-free or compartmentless substrate) of the LFA. In some examples, the bioelectrochemical cell may be a piece of paper that has been impregnated with a solution of glucose and a redox-species (e.g., potassium ferricyanide K₃[Fe(CN)₆]³⁻, or ferrocene or a ferrocene derivative) and dried. In some examples, the media (e.g., membrane, paper, and/or substrate) of the lateral flow immunoassay device can act as a bioelectrochemical cell, as further described below. Additionally, examples disclosed herein label (e.g., attach, couple, etc.) an enzyme (e.g., glucose oxidase (GOx)) to antibodies analytes, and/or antigens on the conjugate pad. In this manner, if the antibodies analytes, and/or antigens coupled to GOx attach to a target analyte, the immobilized antibodies, analytes, and/or antigens corresponding to the target analyte at the test zone attaches and immobilizes the GOx. Because one half of the bioelectrochemical cell is attached to the test zone, the GOx reacts with the solution of the bioelectrochemical cell to oxidize the glucose of the bioelectrochemical cell as shown in the below chemical reaction Processes 1-3, where (Ox) and (Red) refer to the reduced or oxidized state of the enzyme, respectively. In some examples (e.g., when the porous media acts as the bioelectrochemical cell), the glucose is introduced as part of a buffer applied by a user. In some examples, the glucose is introduced in a holding component (e.g., a blister, a stamp, a bag, a sack, etc.) as further described below.

Glucose+GOx(Ox)⇒Gluconolactone+GOx(Red) (Process 1)

GOx(Red)+O₂⇒GOx(Ox)+H₂O₂ (Process 2)

Gluconolactone+H₂O⇒Gluconic Acid (Process 3)

Additionally, the below Processes 4-5 illustrate chemical reactions, where cofactor flavin adenine dinucleotide (FAD) and FADH₂ refer to the oxidized or reduced state of the enzymes active center, respectively.

GOx(FADH₂)+O₂⇒GOx(FAD)+H₂O₂ (Process 4)

GOx(FAD)+Glucose⇒GOx(FADH₂)+Gluconolactone (Process 5)

Although examples disclosed herein refer to GOx as the enzyme used to generate a product (e.g., hydrogen peroxide (H₂O₂)) that corresponds to current flow and/or release/movement of electrons, examples disclosed herein may be implemented using additional and/or alternative enzymes. Examples disclosed herein may utilize amino acid oxidase, Nicotinamide adenine dinucleotide phosphate (NADPH) oxidase, alcohol oxidase, galactose oxidase, and/or any other oxidase as an enzyme used to generate a product (e.g., hydrogen peroxide) that facilitates a release and/or movement of electrons. In some examples, based on the enzyme used, molecules other than glucose are included to facilitate the reaction to generate hydrogen peroxide.

In some examples, an enzymatic reaction with a natural mediator (electron acceptor) (e.g., oxygen) can be used for a bioelectrochemical cell. In such an example, glucose oxidizing to gluconolactone and FADH₂ oxidizing to FAD, thereby resulting in a product (e.g., H₂O₂ (hydrogen peroxide)). Additionally or alternatively, glucose is oxidized by oxygen (e.g., oxygen is reduced) in the presence of glucose oxidase to cause C₆H₁₂O₆ and oxygen (O₂) to react and generate C₆H₁₀O₆ and H₂O₂ (e.g., hydrogen peroxide). In some examples, the electrodes may be made of a metal (e.g., copper, titanium, brass, silver, platinum, etc.) graphite, or a screen printed carbon electrodes doped with ferrocyanide. In this manner, the product (e.g., H₂O₂) is reduced and oxidation of the metal or ferrocyanide of the electrode occurs by release of electrons. In later case ferrocyanide [Fe(CN)₆]⁴⁻ reacts to [Fe(CN)₆]³⁻. Released electrons can be measured by a processor (e.g., using a current and/or voltage measurement). Copper surface is normally oxidized by air to Cu₂O(Cu(I)). Cu₂O is oxidized to CuO(Cu(II)) by reduction of H₂O₂.

In other examples, alternative reactions may be implemented with a bioelectrochemical cell. For example, an amperometric redox-reaction without enzymes can be used for a bioelectrochemical cell. For example, an amperometric signal of an LFA can be measured without the GOx enzyme because the gold of the AuNP may act as a catalyzer (e.g., where the GOx in the conjugate is replated with AuNP). In such examples, thiosulfate may be used to improve the signal as shown below in Processes 6-8. In the below Process 11, thiosulfate, ferricyanide, and KBr or KCl react and the AuNP will catalyze reduction to the ferrocyanide to create an electronic signal. The KBr or KCl may be included in the buffer and/or may be dried in a portion of the porous membrane and resuspended with a buffer.

(Oxidation) 2S₂O₃²⁻⇒S₄O₆²⁻+2e⁻ (Process 6)

(Reduction) [Fe(CN)₆]³⁻+e⁻⇒[Fe(CN)₆]⁴⁻ (Process 7)

2[Fe(CN)₆]³⁻+2S₂O₃²⁻⇒2[Fe(CN)₆]⁴⁻+S₄O₆²⁻ (Process 8)

Alternatively, the below Processes 9-11 illustrate alternative chemical reactions for GOx-glucose oxidation. As shown in the previous processes, oxygen can re-activate or re-oxidize GOx to its active form. Although Processes 9-11 are described in conjunction with ferricyanide, the Processes 9-11 may be used with other substances in their oxidized form (e.g., quinones, ferrocenes, osmium complexes, etc.). The quinones, ferrocenes, osmium complexes, etc. may be included in the buffer and/or may be dried in a portion of the porous membrane and resuspended with a buffer.

Glucose+GOx(Ox)⇒Gluconolactone+GOx(Red) (Process 9)

GOx(Red)+2[Fe(CN)]³⁻⇒GOx(Ox)+2[Fe(CN)]⁴⁻ (Process 10)

Gluconolactone+H₂O Gluconic Acid (Process 11)

Processes 4 and 11 result in a decrease in the pH level of the solution (e.g., to acidic regime in case of a low buffering system), which corresponds to an electromotive force with respect to an electrode placed next to a test line of an LFA, where a GOx reaction does not occur. A change of the pH level will occur in low concentrated buffer solutions and results in a measurable Nernst voltage and/or current.

Within a redox-cycle of GOx, an electron can be transferred to reduce [Fe(CN)₆]³⁻ to [Fe(CN)₆]⁴⁻ at one half of the bioelectrochemical cell. The ferricyanide molecule acts as an electron mediator and can finally diffuse to the electrode. The concentration difference of an electrode at the upper portion of a cell and an electrode at the bottom portion of the cell creates a cell voltage described by the Nernst equation

$$E = 0.0 \; 59 V + log \frac{{[ {Fe ( CN )}_{6} ]}^{3 -}}{{[ {Fe ( CN )}_{6} ]}^{4 -}} and / or$$

$$E = 0.059 V + log \frac{{[ {H}_{3} {O}^{+} ]}_{cell \_ bottom}}{{[ {H}_{3} {O}^{+} ]}_{cell \_ top}} .$$

Accordingly, the porous media in conjunction with the cell and/or the electrode generate an electrical signal (e.g., a voltage or current) when a target analyte is present in a sample. Examples disclosed herein measure a voltage and/or current from a cell (e.g., from one half to the other half) corresponding to current through the potentiometric cell to determine whether the test is positive (e.g., the corresponding target analyte is present in a sample) or negative (e.g., the corresponding target analyte is not present in a sample), thereby eliminating the need for a visual indicator. There may be other combinations of elements and/or reduction agents other than the examples listed above that may be additionally or alternatively used when implementing a bioelectrochemical cell. An LFA that leverages a reaction that results in detection of ions in a solution based on electric current or charges in electric current (e.g., an amperometric measurement) is herein referred to as an amperometric-based LFA. An LFA that leverages a detection of an electric potential difference between two electrodes (e.g., a potentiometric measurement resulting from a redox reaction) is herein referred to as a potentiometric-based LFA.

As described above, the media (e.g., membrane, paper, and/or substrate) of the lateral flow immunoassay device can act as a bioelectrochemical cell. For example, instead of using a paper dried with glucose and a redox species to act as a bioelectrochemical cell. A liquid assay buffer may be applied to the lateral flow immunoassay device that includes a redox species and the glucose is included in a component that releases the glucose at and/or near the test lines and/or control lines at a particular point in time. In some examples, the buffer can dilute the sample and include a redox species to facilitate the reaction that results in a current or voltage. In such examples, the membrane of the lateral flow immunoassay device acts as the salt-bridge. In this manner, if the immobilized antibodies analytes, and/or antigens corresponding to the target analyte at the test zone attaches immobilize the GOx, the GOx will react with the released glucose from the component that holds the glucose and redox species of the assay buffer to diffuse electrons to a zone of the media outside of the test zone, thereby creating a voltage and/or current. Accordingly, the bioelectrochemical cell is based on the test zone and a zone outside the test zone. Thus, a first electrode can be placed at the test zone and a second electrode can be placed at a zone outside of the test zone and a voltage and/or current can be measured between the two test zones to determine if the target analyte is present. As used herein, a bioelectrochemical cell is a cell that converts chemical energy of a fuel and an agent (e.g., an oxidizing agent) into electricity through a pair of redox reactions. A bioelectrochemical cell could be a biofuel cell (e.g., a fuel cell that uses enzymes as a catalyst to oxidize its fuel), a concentration cell (e.g., an electrolytic cell including two half-cells with the same electrodes that creates a voltage and/or current when the two half-cells have different concentrations (e.g., GOx Glucose reaction at one electrode and no reaction at other electrode)), a galvanic cell (e.g., including two different metals immersed in electrolytes connected by a salt bridge or porous media), and/or any other cell that converts chemical energy into electricity.

Because the LFA device may correspond to one or more specific durations of time that a user should take a particular action (e.g., a duration of time to apply the sample and/or a buffer, a duration of time to obtain results after applying a sample, a duration of time to scan the LFA device with a reader, a duration of time to move a mechanical piece of the LFA device, etc.), examples disclosed herein including mechanisms and processes to track time and/or guide a user to perform one or more steps. For example, after a sample and/or buffer is applied to an LFA, the user may need to wait for a first duration of time before obtaining the results. Examples disclosed herein may include a timer at the LFA device and/or at the reader to track the duration(s) of time to guide the user through the testing process. In some examples, the timer is powered by the current and/or voltage generated by the bioelectrochemical cell. In some examples, the LFA device may transmit the timing information to the LFA reader and the LFA reader may display and/or otherwise indicate one or more durations of time to the user. In some examples, the LFA reader may track durations of time based on confirmations from the user and guide the user as to when to perform particular tasks to ensure that a task is not performed too early or too late (e.g., reading a result too early or too late, scanning the LFA too early or too late, adjusting the LFA device too early or too late, etc.). By providing a reader to guide a user through the testing product, LFA devices can be operated by the user with less training and/or may be performed by a user with less education level. Additionally, the reader can be implemented by the patient for self-testing by following the guide presented on the reader.

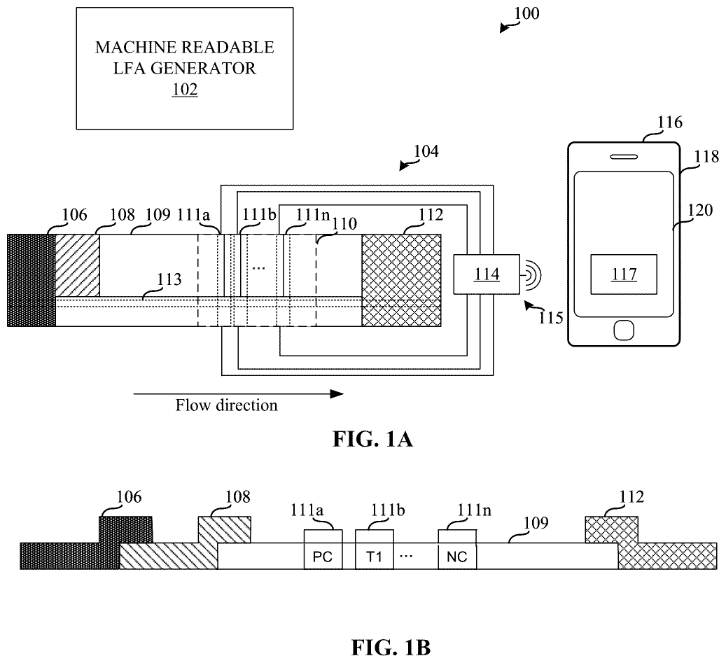

FIG. 1A is an example environment 100 including an example machine readable LFA generator 102 to generate an example machine readable LFA chip, strip, or LFA device 104 (illustrated in an overhead view). The example machine readable LFA device 104 includes an example sample pad 106, an example conjugate release pad 108, an example porous media 109, an example test area 110, example bioelectrochemical cells 111a-n, an example wicking pad 112, an example inlet 113, an example wireless chip 114, and an example antenna 115. The environment 100 of FIG. 1A further includes an example user device or reader 116 to determine the test results (e.g., diagnostic test results) of a sample being applied to the example machine readable LFA device 104. The example reader 116 includes an example machine readable LFA reader application 117, an example antenna 118, and an example user interface 120. Although the example of FIG. 1A determines test results based on the bioelectrochemical cells 111a-n, FIG. 1A may be described in conjunction with other techniques for determining test results based on an electrical signal, as further disclosed herein.

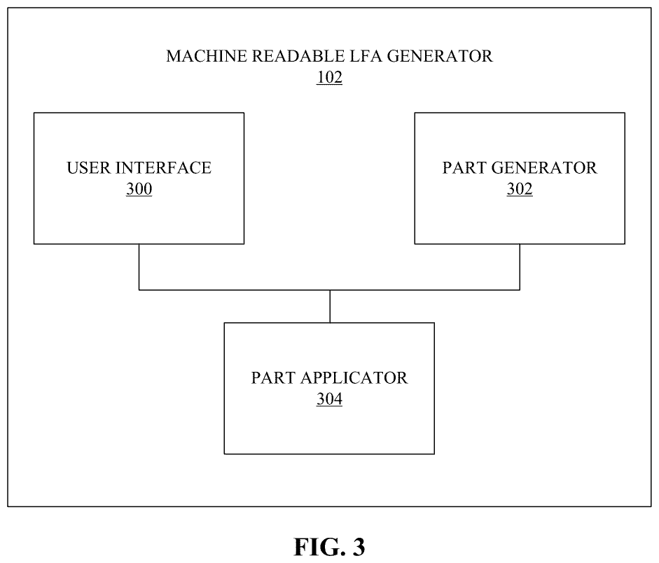

The example machine readable LFA generator 102 of FIG. 1A generates the example machine readable LFA device 104. For example, the machine readable LFA generator 102 generates the machine readable LFA device 104 to include the example sample pad 106, the example conjugate release pad 108, the example porous media 109, the example test area 110, the example bioelectrochemical cells 111a-n, the example wicking pad 112, the example inlet 113, the example wireless chip 114, the example antenna 115, and/or wires or etches included in a housing structure (not shown). The number of test, types of test (e.g., pregnancy, Ebola, HIV, influenza, covid, cancer, sexually transmitted diseases (STDs), Covid-19, etc.), number of control zones, types of control zones (e.g., positive control, negative control, etc.), and/or type of machine readable LFA is based on instructions from a user and/or manufacture. For example, if a user wants the example machine readable LFA device 104 to include a single test for pregnancy, the machine readable LFA generator 102 generates the example conjugate region to include antibodies, analytes, and/or antigens corresponding to a target analyte that corresponds to pregnancy, the antibodies, analytes, and/or antigens labelled with a particular molecule (e.g., gold nanoparticles, GOx, etc.). In such an example, the machine readable LFA generator 102 generates the test area 110 to include immobilized antibodies, analytes, and/or antigens to bind or otherwise attach to the target analytes). Additionally, the example machine readable LFA generator 102 generates, or otherwise attaches, the wireless chip 114 to attach to the bioelectrochemical cell to determine whether a sample (e.g., a biological sample) is positive or negative for the target analytes corresponding to pregnancy. The example machine readable LFA generator 102 is further disclosed in conjunction with FIG. 3.

The example machine readable LFA device 104 of FIG. 1A is a bioelectrochemical cell-based LFA (e.g., a concentrations cell with Nernst voltage sensing) that includes the example bioelectrochemical cells 111a-n. However, the machine readable LFA device 104 may be alternative devices that can obtain data related to a test result on the LFA 104. In some examples, the example machine readable LFA device 104 is a non-visual indicating biosensor device. For example, the LFA device 104 may not output a visual indication to a user (e.g., because no gold nanoparticles are present on the device) and/or the visual indication is not visible to a user (e.g., because the housing covers the porous media where the visual indications may be implemented). In some examples, the LFA device 104 is a visual indicating biosensor device that provides a visual indication of the results and is capable of transmitting the results wirelessly to a reader. The LFA device 104 may be a point-of-care device and/or a rapid diagnostic device containing an application-specific integrated circuit (e.g., the example wireless chip 114).

The example machine readable LFA device 104 of FIG. 1A is a device that includes the sample pad 106 (e.g., a sample pad, a sample region, a sample area, a sample zone, etc.). For example, the LFA device 104 may be a porous membrane device, a porous media device, a fluid transporting media device, a test strip device, a lateral flow test strip device, and/or any fluid sample device. The sample pad 106 is structured to act as a sponge to hold a sample of fluid applied to the sample pad 106. In some examples, the sample pad 106 includes buffer components (e.g., salts, surfactants, etc.) to ensure that target analytes that may be in the sample are capable of binding with components of the conjugate release pad 108. When the sample pad 106 is soaked, the fluid held in the sample pad 106 flows to the conjugate release pad 108. The conjugate pad 108 includes a labeled substance or conjugate configured to bind a target analyte. For example, the conjugate release pad 108 includes conjugates or probes (e.g., antibodies specific to one or more target analyte) labeled detectable labels, tags, linkers, antibodies, analytes, antigens, GOx, gold nanoparticles, etc. The target analyte is a component that corresponds to a particular condition or disease. Accordingly, presence of the target analyte in the sample corresponds to presence of the corresponding condition and/or disease in the patient who provided the sample. If the sample includes the one or more of the target analytes, the conjugates and/or probes labelled with the GOx, gold nanoparticles, etc., attach to the corresponding target analytes. The sample (e.g., including the probes if corresponding target analytes are present in the sample) continues to flow through the example porous media 109 of the machine readable LFA device 104 toward the wicking pad 112.

While the sample flows across the media 109 of FIG. 1A, the sample flows across the test area 110. The media 109 may be a porous membrane, a nitrocellulose membrane, a paper, and/or other substrate including a compartment-free substrate, etc. that propagates the flow of the biological sample and/or liquid buffer. The test area 110 includes test zones (e.g., test lines, test area, test region, etc.) and/or control zones (e.g., control lines, control area, control region, etc.) that are displaced a longitudinal axis of the media 109. The test zones include specific immobilized antibodies, analytes, or antigens that react with the corresponding target analytes attached with the probes and/or conjugates. Accordingly, when the target analyte corresponding to a particular test zone is present in the sample, the GOx and/or gold nanoparticles immobilize at the particular test zone. In some examples, the test zones of the LFA 104 corresponds to different conditions or diseases, known as multiplexing. Multiplexing includes structuring multiple test zones or test lines to detect multiple marks (e.g., multiple types of antibodies/antigens/analytes or multiple analytes) in one sample. Multiplexing has applications, for example, in a single diagnostic test that could test for multiple types of venereal diseases. In some examples, the test area 110 includes control zones that immobilize specific, and/or excess, conjugates flowing past the control zones. The control zones correspond to targets which have been dried to the porous media 109 to signify that the test is ready to be read (e.g., when the test is complete).

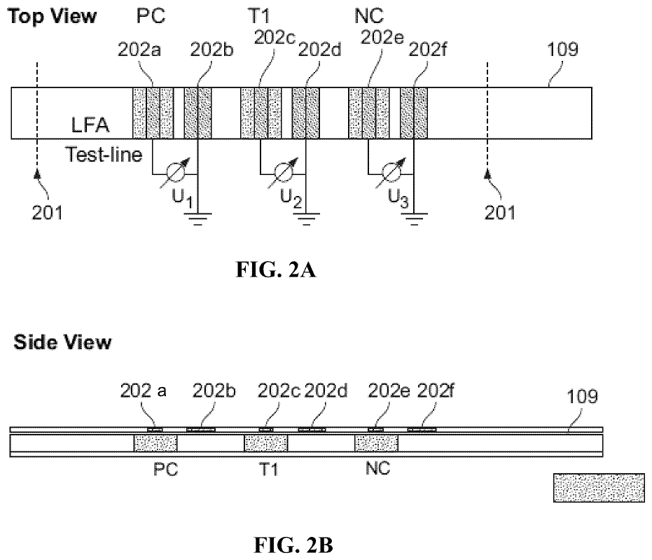

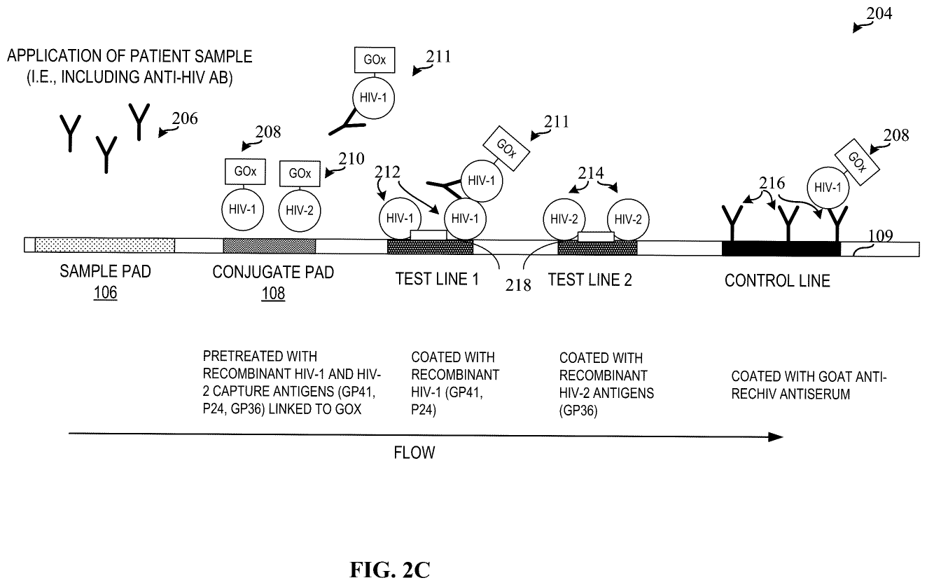

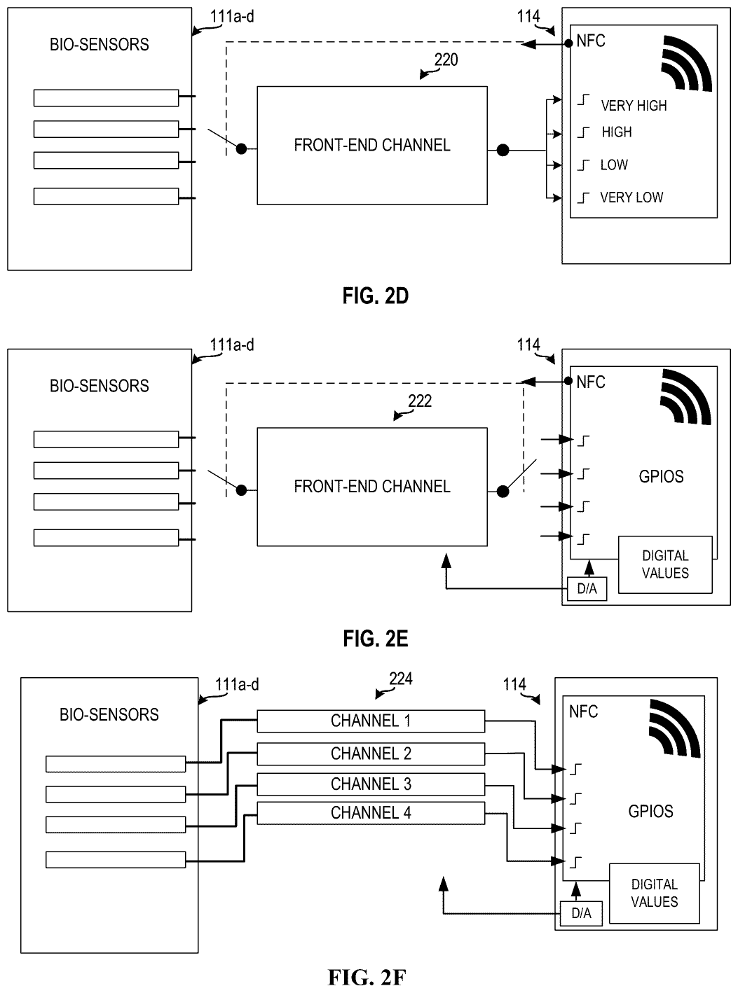

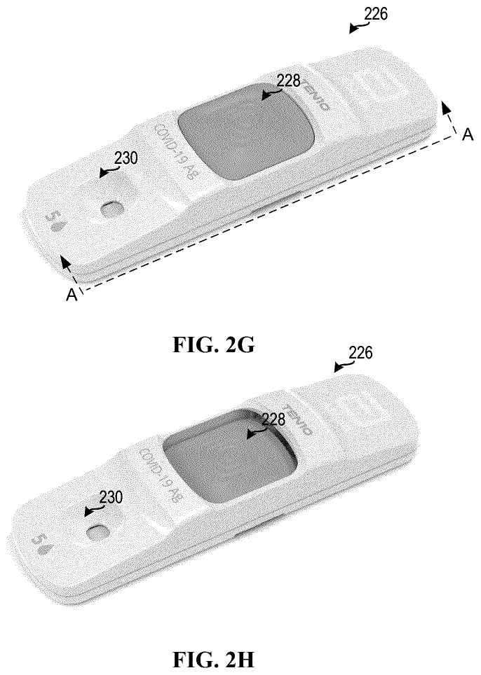

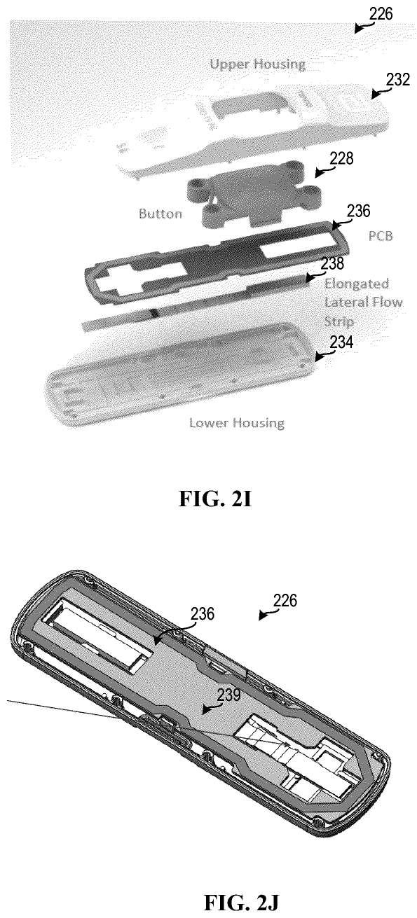

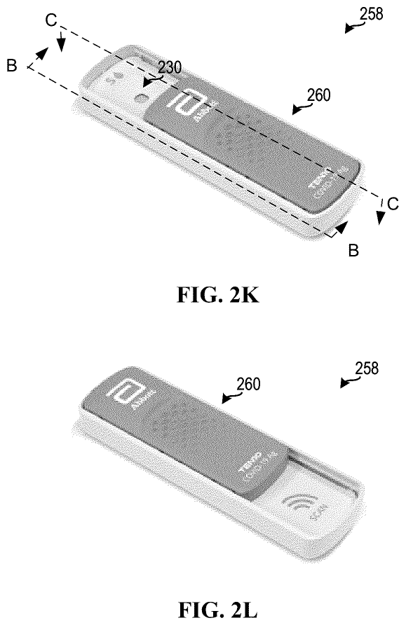

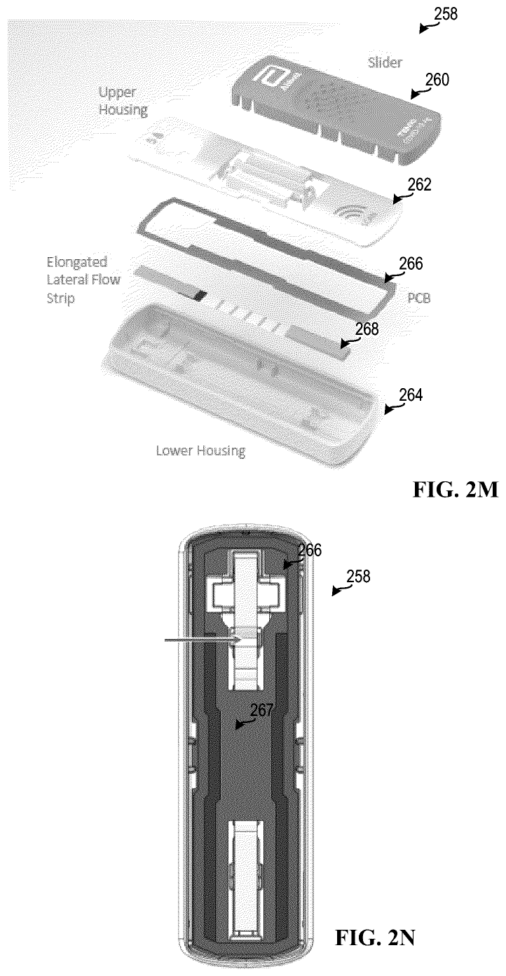

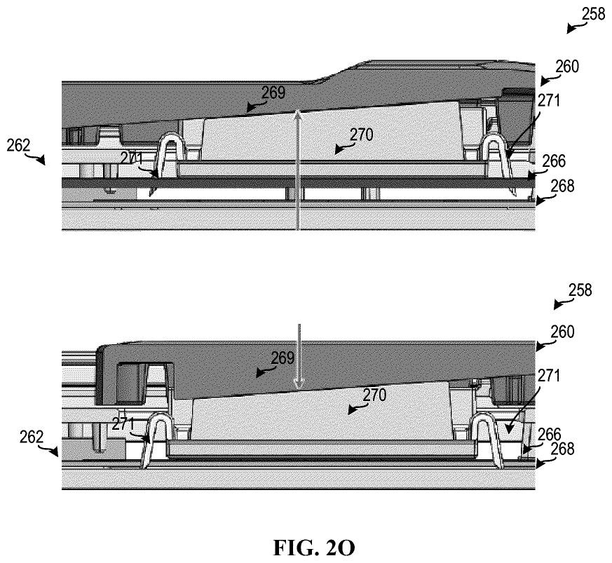

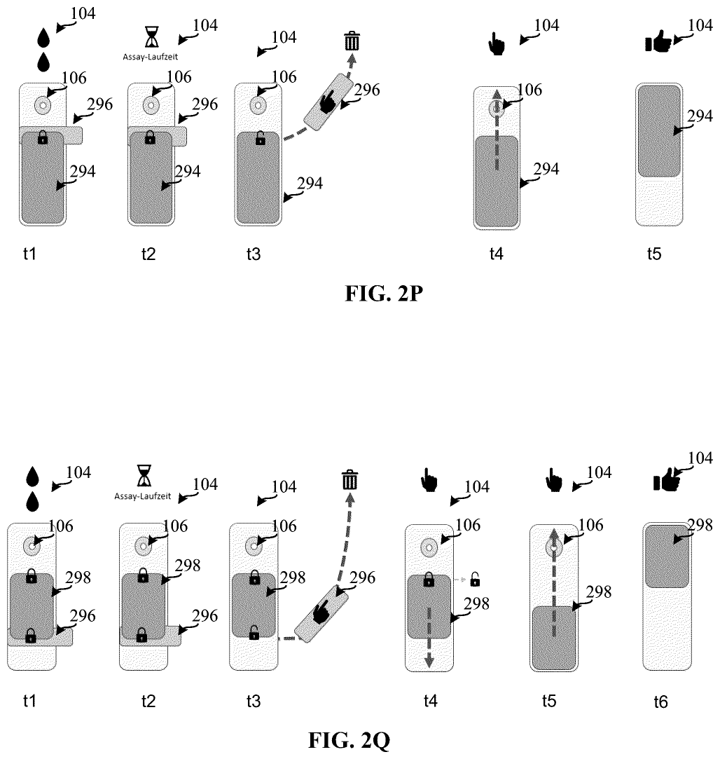

The test area 110 of FIG. 1A further includes the example bioelectrochemical cells 111a-n (e.g., concentration cells). FIG. 1A illustrates a portion of the bioelectrochemical cells 111a-n. In some examples, the potentiometric cells 111a-n are pieces of paper or other substrate impregnated with an enzyme substrate (e.g., glucose) and/or a reducing agent (e.g., hydroquinone, aminophenol, ascorbic acid included in vitamin C, etc.), and/or an electron mediator or a redox-species (e.g., potassium ferricyanide, ferrocene, oxygen, a ferrocene derivatives, etc.). However, the bioelectrochemical cells 111a-n can be any type of bioelectrochemical cell. For example, the porous membrane 109 can act as one or more bioelectrochemical cells, as further described below in conjunction with FIGS. 2A-2B. When GOx (which oxidizes glucose) or gold nanoparticles (silver nitrate reduction (e.g., vitamin C and/or another reducing agent) via autocatalysis) attaches to the test zones and/or control zones, the corresponding bioelectrochemical cells 111a-n are activated by generation of a voltage according to the Nernst equation, which can be read out via the wireless chip 114. Accordingly, the wireless chip 114 uses example bioelectrochemical cells 111a-n to perform a bioelectrochemical measurement, where the wireless chip 114 passively measures the potential between two different parts of the biofuel cells 111a-n using two electrodes. In some examples, the bioelectrochemical cells 111a-n are fluidically coupled (e.g., attached) to the test and/or control zones during manufacturing. In some examples, the bioelectrochemical cells 111a-n are separate from the test and/or control zones during manufacturing and the example LFA device 104 includes a mechanical component and/or device to automatically and/or manually press the bioelectrochemical cell into contact (e.g., fluidically contact) with the test and/or control zones when a test is to be performed (e.g., automatically when a sample is applied to the LFA 104 and/or manually via a user interference to press the bioelectrochemical cell into contact with the zones).

The test area 110 of FIG. 1A further includes the example wicking pad 112. The example wicking pad 112 (also referred to as a waste pad) is an absorbent material that wicks the liquid through the LFA. In some examples, the wicking pad 112 includes cellulose filters. The wicking pad 112 prevents backflow of the liquid. Also, in some examples, the wicking pad 112 acts as a waste container. As used herein, downstream is referred to as toward the waste pad 112 and upstream is referred to as toward the sample pad 106.

The test area 110 further includes the inlet 113 of FIG. 1A. The example inlet 113 of FIG. 1A is structured to allow water or another buffer solution applied to the sample pad 106 to flow to the wicking pad 112. In some examples, the water and/or buffer is enclosed in a holding device (e.g., a frangible enclosure such as a bag) and the user presses and/or moves a component of the LFA device 104 to cause the holding device to release the buffer (e.g., by breaking the bag). In some examples, the sample pad 106 may be more than one sample pads (e.g., one for the sample and another for the water/buffer and/or further reagents). When the water and/or buffer flows past the bioelectrochemical cells 111a-n, the water and/or buffer resuspends the dried reagents to cause the current to flow if the test and/or control zone has attached to GOx and/or gold nanoparticles (e.g., based on the mixing of the conjugate with the enzyme substrate, the reducing agent, and/or the electron mediator of the bioelectrochemical cells 111a-n). A center area (e.g., the constricted area in FIG. 2A) between a first portion (e.g., the top portion in FIG. 2A) and a second portion (e.g., the bottom portion in FIG. 2A) of the bioelectrochemical cells 111a-n acts as a salt-bridge against diffusion induced homogenization of a concentration gradient, thereby causing electrons lost in the first portion of the bioelectrochemical cells 111a-n (e.g., due to the reactions that occur when the target analyte and corresponding conjugate is suspended in the test zone or control zone) to flow toward the second portion of the bioelectrochemical cells 111a-n, corresponding to a measurable voltage drop and/or current from the first portion of the bioelectrochemical cells 111a-n to the second portion of the bioelectrochemical cells 111a-n. Additionally or alternatively, the paper/membrane type could reduce diffusion.

The example machine readable LFA device 104 further includes the example wireless chip 114. The wireless chip 114 is a near field communication (NFC) chip. Accordingly, the wireless chip 114 can obtain power and/or communicate with the reader 116 via NFC communication protocols. Alternatively, the example wireless chip 114 may be a radio frequency identification (RFID) chip or any other type of wireless chip. The wireless chip 114 includes pins coupled to connectors (e.g., wires, etches, etc.) that couple to electrodes (e.g., directly or via a front-end device, as further described below). The electrodes connect to the two ends of each bioelectrochemical cell 111a-n and/or on different sections of the porous membrane 109. In this manner, if voltage and/or current is generated by the bioelectrochemical cell 111a-n, a voltage drop and/or current between the two electrodes (e.g., connected to the opposite sites of the bioelectrochemical cell or placed in different locations on the porous membrane 109) will be higher than a threshold. In some examples, the generated voltage and/or current can be used to power the wireless chip 114 (e.g., to provide power for storing the results of the test at the wireless chip 114). The example wireless chip 114 includes hardware, software, and/or firmware capable of measuring a current, an electromotoric force, or the voltage drop and/or current to determine if a target analyte is present in a sample. The wireless chip 114 may include an ASIC to encode manufacturing and/or identification information. The example wireless chip 114 is powered by the example antenna 115. When the example reader 116 generates a magnetic field within a threshold distance to the example antenna 115, a current is generated at the antenna 115 and the corresponding energy is used to power the wireless chip 114. The energy can be used to provide a voltage to power the wireless chip 114. In some examples, the voltage is (e.g., 1.8 Volts (V)). In other examples, the voltage may be of a different magnitude including, for example, 3.3 V, 5 V, etc. Once powered, the wireless chip 114 can operate. For example, in operation the wireless chip 114 can apply potentials. In some examples, the wireless chip 114 operates to measure voltages, currents, and/or resistances, etc. Additionally or alternatively, in some examples, the wireless chip 114 operates to transmit identification information (e.g., device identifier, test identifier, serial number, product code, etc.) to the example reader 116 and/or may transmit measurements corresponding to test results (e.g., analog and/or digital values corresponding to voltage and/or current measurements taken at the LFA device 104). In some examples, the communication between the wireless chip 114 and the reader 116 is encrypted using an encryption technique known by both the wireless chip 114 and the reader 116. In some examples, the wireless chip 114 may flag each test zone and/or control zone as positive or negative based on the measured current, electromotoric force, and/or voltage drop. Also, in some examples, the wireless chip 114 stores the results corresponding to the flag(s), and/or uses the antenna 115 to transmit the results. Additionally, the example wireless chip 114 may transmit identification information corresponding to an LFA identifier, identifiers for the test and/or control zones, etc., with the results. Although the example of FIG. 1A includes the example wireless chip 114 as part of the example LFA 104, the wireless chip may be integrated in an external device. In such examples, the LFA 104 may include a component (e.g., an interface) that allows the electrodes of the LFA 104 to connect to the wireless chip 114. In this manner, design of the LFA 104 can be simplified to reduce the size and/or cost of the LFA 104 and multiple LFAs can be connected to the wireless chip 114 to transmit test results to the example reader 116.

The example reader 116 of FIG. 1A is a smartphone that includes the machine readable LFA reader application 117, the example antenna 118, and the example user interface 120. Alternatively, the example reader 116 may be a tablet, a personal digital assistant, a laptop, a standalone LFA reader device, and/or any other processing device that includes, or is otherwise in communication with, the example machine readable LFA reader application 117, the example antenna 118, and/or the example user interface 120. The example machine readable LFA reader application 117 is an application that can be installed, downloaded, and/or coded within the example reader 116. In some examples, the example machine readable LFA reader application 117 can be supplied via a near field communication tag or Bluetooth device.

In some examples, the reader 116 can be split into multiple readers. In such examples, a first reader 116 obtains the information from the LFA 104 and guides the user through the process with the LFA 104, and a second reader 116 obtains the test results from the LFA 104. For example, in a hospital, a hospital information system, a LIMS system, a stock consumption monitor, a clinic, a testing site, etc., the first reader may guide a first user to use a first LFA reader to get samples, once the samples are taken, the first reader may communicate with the second reader to send the LFA device information and initiate the timer at the second reader for the when the test is ready. In this manner, the first user can provide the LFA device to the second user and the second user can scan the LFA devices when ready using the second reader.

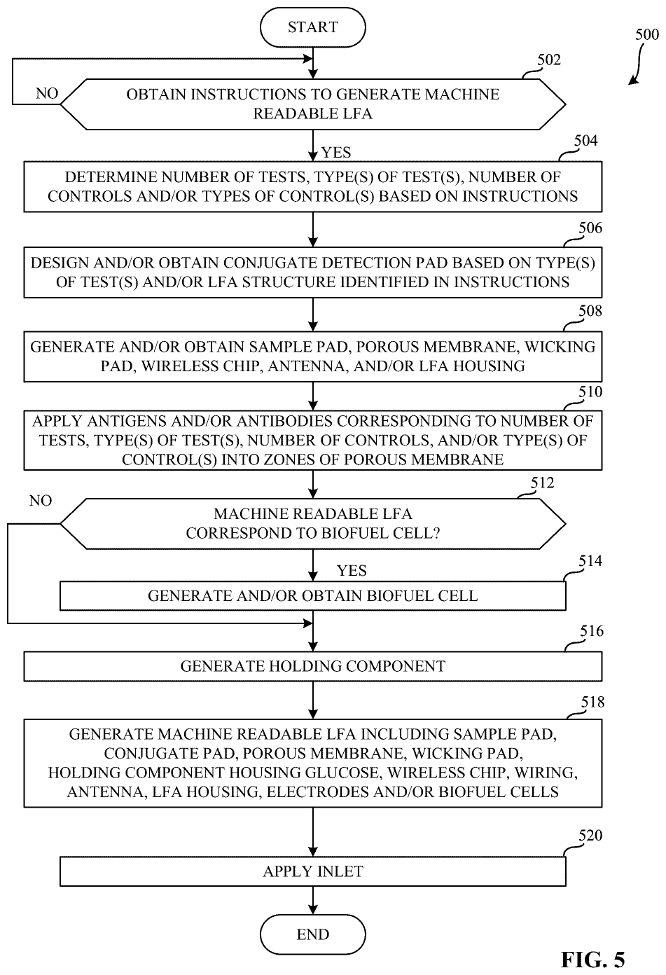

The example machine readable LFA reader application 117 of FIG. 1A instructs components of the reader 116 to generate an electromagnetic signal at various points in time to (a) obtain identification information from the LFA device 104 at a first time, (b) obtain the identification information from the LFA device 104 a second time prior to obtaining the results to verify that the correct device is being read, and (c) obtain the results (e.g., one or more digital voltages and/or currents corresponding to the results) from the LFA device 104. The example LFA reader application 117 causes the electromagnetic field to be generated a second time to verify the identification information to ensure that the user is reading the test from the correct LFA device (e.g., when reading multiple tests concurrently). The example machine readable LFA reader application 117 identifies the results of an LFA-based test by interacting with the wireless chip 114 (e.g., receiving a wireless signal identifying the results of a test). For example, the machine readable LFA reader application 117 controls the components of the reader 116 to obtain identification information corresponding to the machine readable LFA device 104 and/or test results via a NFC, RFID, etc. signal transmitted from the wireless chip 114. In some examples, the LFA reader application 117 determines information based on the obtained identification information. For example, the LFA reader application 117 can determine if the test has already been read, the type of test that is being performed, an algorithm for identifying and/or categorizing the results of the test, whether the test has expired or recalled, etc. The algorithms (e.g., corresponding to how and when to read one or more electrical signals from a particular LFA device) may be stored locally at the reader 116 and/or may be configured, reconfigured, updated, etc. remotely (e.g., via a patch, an update, remote instructions, etc.). Once the test results are obtained (e.g., the samples from the LFA device 104), the machine readable LFA reader application 117 determines if the test is ready (e.g., based on the flags of corresponding to the control zones) and, if ready, determines the results of the test based on a corresponding algorithm (e.g., using voltage and/or current samples from the test lines) and displays the results using the example user interface 120, stores the results in a local database, and/or transmits the results to a monitoring entity for monitoring and/or statistical analysis. In this manner, the manufacturer or other party can process the results, perform diagnostics, and/or identify if a particular LFA device may be counterfeited (e.g., if more than a threshold number of results corresponding to the same identification information has been determined). In some examples, the LFA reader application 117 transmits raw data. In some examples, the LFA reader application 117 obtains (e.g., from the wireless chip 114) and/or determines (e.g., based on identification information from the wireless chip 114) various other contextual information including lot number, expiration date, expiry date, test information, signal quality information, chain of custody, etc. Additionally or alternatively, in some examples, the LFA reader application 117 transmits processed data or data resulting from one or more levels of analysis. The example machine readable LFA reader application 117 is further disclosed below in conjunction with FIG. 5.

The example antenna 118 of the example reader 116 of FIG. 1A wirelessly powers and/or communicates with the example wireless chip 114 (e.g., via the example antenna 115). For example, based on instructions from the example machine readable LFA reader application 117, the antenna 118 emits a magnetic field to power the example wireless chip 114 and receives wireless information (e.g., identifiers, test results, etc.) from the example wireless chip 114.

FIG. 1B is a side view of the LFA components of the LFA device 104 of FIG. 1A. FIG. 1B includes the example sample pad 106, the example conjugate pad 108, the example porous media 109, portion(s) of the example bioelectrochemical cells 111a-111n, and the example wicking pad 112 of FIG. 1A. The example bioelectrochemical cells 111a-n are connected to the example wireless chip 114 of FIG. 1A via electrodes, as described above in conjunction with FIG. 1A.

FIG. 2A illustrates a top view of an example implementation of the porous media 109 of the machine readable lateral flow immunoassay 104 of FIG. 1A. In the example of FIG. 2A the membrane 109 acts as the bioelectrochemical cell 111a-n of FIG. 1A. The example of FIG. 2A further includes two control lines (e.g., positive control (PC) and negative control (NC)), a test line (T1), and example electrodes 202a-202f. Although the example porous media 109 has two control lines, one test line, and six electrodes, the porous media 109 may have any number of test lines, control lines, and/or electrodes.

The negative control line includes immobilized antibodies and/or antigens to couple to corresponding target analytes that couple to the immobilized antibodies when a sample is applied to the sample pad 106. The negative control line is included to verify that the current generated at the corresponding electrodes 202e, 202f is strong enough for a valid test measurement. For a test to be valid, the amount of voltage and/or current across the electrodes 202e, 202f over the negative control line should be lower than a threshold.

The positive control line includes immobilized antibodies, analytes, and/or antigens that couple to specific, and/or excess, conjugates labelled with GOx and/or gold nanoparticles and/or probes labelled with GOx and/or gold nanoparticles flowing from the conjugate pad 108 toward the wicking pad 112. When a sample flows toward the wicking pad 112, the positive control line is the last section to attach to the conjugates and/or probes, resulting in a voltage and/or current generation between the electrodes 202a, 202b. In this manner, the reader 116 knows if the test is ready when the result of a voltage and/or current measurement across the electrodes 202a, 202b results in positive (e.g., more than a threshold amount of voltage and/or current). The example test line T1 has immobilized antibodies, analytes, and/or antigens that correspond to a target analyte coupled to the porous media or medium 109. Accordingly, if the target analyte for T1 is present in a sample, the target analyte (which is attached to a conjugate antibody, analyte, and/or antigen labeled with GOx and/or silver nitrate) immobilizes at the T1 line.

Although the example electrodes 202a-f of FIG. 2A have different widths, the example electrodes 202a-f can be the same width and/or be structured to be any width. The width of the electrodes 202a-f may be based on the geometry of the porous membrane 109, the size of the salt bridge in between the working electrode (e.g., electrode 202a) and reference electrode (e.g., electrode 202b), overlap with specific test zone and out reference areas, etc. In some examples, the electrodes 202a, 202c, 202e are wider than the electrodes 102b, 102d, 102f (e.g., reference electrodes) and the electrodes 202a, 202c, 202e are wider than the test-bands in order to allow most flexible positioning tolerance for assembly and during manufacturing/printing of test-bands. Spacing between working-electrode to counter-electrode pairs [electrode 202a, reference electrode 202b], [electrode 202c, reference electrode 202d], [electrode 202e, reference electrode 202f] are as small as possible, (e.g., 10 nanometers (nm) to 5 millimeters (mm), but could be as small as 1 nm). The example electrodes 202a-202f may be silver carbon electrodes, copper electrodes, graphite carbon electrodes, titanium electrodes, brass electrodes, platinum and palladium electrodes, screen printed carbon electrodes doped with ferrocyanide or any other oxidizable material or electron donator, and/or any other type of electrode. In some examples, the electrodes 202a, 202c, 202e are copper electrodes and the reference electrodes are carbon electrodes. By having a pair of electrodes (e.g., a working electrode and a reference electrode) for a single test line and/or control line, a salt bridge can be created on the porous membrane 109 between the pair of electrodes with a low amount of impedance. In this manner, a voltage and/or current between the pair of electrodes can be read without using an external power source to facilitate the generation of the voltage and/or current. In some examples, the electrodes 202a-202f are not engaged with the porous media 109 until it is time to read a result, as further described below. Accordingly, one or more mechanical devices may be used to keep the electrodes 202a-202f disengaged from (e.g., not in contact with) the porous media 109 until it is time to read the result.

In some examples, a voltage regulator and/or driver of the wireless chip 114 may apply an electric potential (e.g., a voltage, a bias, etc.) across one or more of the electrodes 202a-202f to aid, facilitate, and/or improve one or more chemical reactions that are to occur on the porous membrane 109. In some examples, the applied electric potential is a poised potential or poise. Applying a potential to the electrode(s) facilitates and/or induces chemical reactions (oxidation and/or reduction) to obtain a stronger signal corresponding to the presence of a target analyte. The stronger signal includes, for example, an increased current signal amplitude, a more stable polarity, a reduced signal spread, a greater signal-to-noise ratio, and/or a higher sensitivity (e.g., from 0.01% to 0.001%). For example, the poise or electrical potential affects the chemical reaction to make more electrons flow when a target analyte is present. For example, the penetration of electrons through the electrode surface for an analyte is related to the applied potential (e.g., leading to qualitative analysis). Accordingly, applying an electric potential forces the reaction from the oxidized form to the reduced form of copper of the electrodes. In some examples, the amount of poise, bias, and/or voltage may force and/or aid in different parts of the chemical reaction. Accordingly, in some examples, one or more poise and/or voltage can be applied to aid in one or more of the chemical reactions that result in a current flow when a target analyte is present. The application of a potential and/or poise results in an increased sensitivity and/or higher signal robustness. As further described below, the driver and/or voltage regulator of the wireless chip 114 adjusts the voltage (e.g., 1.8 V, 3.3 V, 5 V, etc.) generated by the antenna (e.g., via the electromagnetic field generated by the reader 116) to one or more smaller voltages (e.g., 150 mV, −100 mV, −200 mV, etc.) that can be used to apply one or more voltages (e.g., poised potentials) to the electrodes 202a-202f, thereby increasing the signal strength for the current/voltage measured when a target analyte is present during a test. For example, the wireless chip 114 may generate a 150 mV voltage by regulating the supply voltage (1.8 V, 3.3 V, 5 V, etc.) to 150 mV and applying the 150 mV to the electrode 202a to facilitate and/or improve the chemical reaction at the test region when a target analyte is present. In this manner, more electrons flow from the test region to the region corresponding to the electrode 202b (e.g., the non-test region) than would flow if a potential were not applied. The potential to improve one or more chemical reactions may correspond to a positive voltage or a negative voltage. The feed (e.g., application) of the potential can be applied via the working electrode or the counter electrode (e.g., corresponding to whether the voltage is positive or negative). Because the wireless chip 114 uses the energy obtained from the antenna 115 via the electromagnetic field, the wireless chip 114 can apply the potential(s) and/or poise(s) without use of a battery.

In the example of FIG. 2A, the porous media 109 itself acts as the salt-bridge of a biofuel cell. A biofuel cell uses an additional enzyme (e.g., reductase, such as laccase) at a counter electrode (e.g., cathode). In such an example, the laccase reduces Oxygen (O₂) with 4e⁻+4H⁺ to 2H₂O from reaction at the anode. The porous membrane 109 generates a voltage and/or current when a target analyte reacts with released enzyme substrate (e.g., glucose) in conjunction with the redox species acting as the electron mediator. In the example of FIG. 2A, the chromatography paper (e.g., impregnated paper of the above-described biofuel cells 111a-d) is not included, an enzyme substrate (e.g., glucose) and/or a redox agent is included in a holding component (e.g., a blister, a stamp, a sac, a bag, etc.), in a buffer, coated on the electrode, etc. In this manner, when the liquid assay buffer including the redox agent is applied to the sample pad 106, the liquid assay buffer saturates the porous media 109. In some examples, after a threshold amount of time, the user and/or a medical technician performs an action on a mechanical component, such as a button (e.g., as further described below in conjunction with FIGS. 2G-2Q), a slider (e.g., as further described below in conjunction with FIGS. 2K-2Q), and/or another interface of a housing of the LFA device to move the electrodes 202a-f into contact with the porous membrane 109, slice the porous media at predefined location(s), and/or break a holding component to release the glucose at the test and/or control zones, as further described below in conjunction with FIGS. 2G-2Q If the target analyte is present in a sample, the corresponding conjugates/probes labelled with an enzyme (e.g., GOx/gold nanoparticles) become immobilized at the corresponding test zone. Because the porous media 109 is saturated and the enzyme substrate (e.g., glucose) and the redox agent are present (e.g., as part of the buffer, coated on the porous media and/or electrodes, included in a blister or other holding compartment, etc.), the enzyme (e.g., GOx) will react with (e.g., oxidize) the enzyme substrate (e.g., glucose) on the test zone while the redox agent acts as an electron mediator to reduce [Fe(CN)₆]³⁻ to [Fe(CN)₆]⁴⁻ at the corresponding test zone. This result corresponds to a measurable voltage drop and/or current between the test zone and non-test zones of the porous media 109, which can be measured by the example wireless chip 114 via the example electrodes 202a-f. Thus, the examples of FIGS. 2A and 2B is a bioelectrochemical cell that is integrated into the LFA. In some examples, when a holding component is utilized, the holding component dissolves after a threshold amount of time of exposure to and/or release of the glucose, as further described below. Because the glucose is released onto the porous membrane after a threshold amount of time, the flow of liquid (e.g., corresponding to the sample and/or buffer) has significantly slowed and/or stopped. Accordingly, the hydrogen peroxide generated at a test and/or control zone will not flow or will flow too slowly to reach a subsequent test and/or control line, thereby reducing and/or eliminating background noise corresponding to hydrogen peroxide bleeding to a subsequent test line and reacting with a subsequent electrode.

In some examples, oxygen may be needed to aid in the generation of the hydrogen peroxide. However, when the electrode(s) 202a-f are placed in contact with the porous media 109, there may not be enough ambient oxygen to facilitate the generation of a sufficient amount of hydrogen peroxide. Accordingly, the electrode(s) 202a-f may include one or more cutouts (e.g., one or more holes of one or more shape(s) and/or size(s)) to provide access to ambient oxygen to generate sufficient hydrogen peroxide when the glucose and GOx react. Additionally or alternatively, the example electrode(s) 202a-f may be shaped to come into contact with the porous media 109 while providing space for ambient oxygen. For example, the electrode(s) 202a-f may be curved, include a waved patter, and/or include any other pattern to be able to both be in contact with the porous membrane 109 and provide a gap and/or pocket to include ambient oxygen that can be used to aid in the generation of hydrogen peroxide.

As described above, in some examples, an enzymatic reaction can occur with a natural mediator (e.g., oxygen) for the bioelectrochemical cell 111a-d. In such an example, glucose oxidizing to gluconolactone and FADH₂ oxidizing to FAD, thereby resulting in H₂O₂. Additionally or alternatively, glucose can oxidize and reduce oxygen in the presence of glucose oxidase to cause C₆H₁₂O₆ and oxygen (O₂) to react and generate C₆H₁₀O₆ and a product (e.g., H₂O₂ (hydrogen peroxide)). In such examples, the electrodes may be made of a metal (e.g., copper) or a screen printed carbon electrodes doped with ferrocyanide. In this manner, the H₂O₂ is reduced and oxidation of the metal or ferrocyanide of the electrode occurs by release of electrons. In later case ferrocyanide [Fe(CN)₆]⁴⁻ reacts to [Fe(CN)₆]³⁻. Released electrons can be measured by a sensor (e.g., using a current and/or voltage measurement) and processed by a processor (e.g., locally at the wireless chip 114 and/or at the reader 116) to determine a test result. Copper surface is normally oxidized by air to Cu₂O (Cu(I)). Cu₂O is oxidized to CuO(Cu(II)) by reduction of H₂O₂.

In addition, the electrochemical measurement is based on diffusion processes between the electrodes 202a-f. Using an intact flow, convection is an additional factor along with the diffusion that affects the current result of the biochemical reaction. Thus, after the product (e.g., hydrogen peroxide) that can react with the electrode to cause current flow is produced at the control and/or test lines of the porous media 109, the product (e.g., hydrogen peroxide) is diluted with the surrounding buffer (e.g., acetate buffer). In this manner, if the electrodes 202a-f come into contact with the porous media 109 after the product has flowed to the wicking pad 112, there may not be sufficient product to react with the metal in the electrodes 202a-f to generate an electrical signal (e.g., via releasing of electrons). Accordingly, examples disclosed herein may arrest (e.g., slow, stop, etc.) the flow (e.g., fully or partially) to prevent and/or slow the product from migrating away from a test zone and/or control zone. In some examples, a mechanical device may be used to arrest the flow by cutting (e.g., fully or partially) the example porous media 109 at the example membrane cuts 201, thereby stopping the product from flowing toward the example wicking pad 112. Additionally or alternatively, flow may be arrested by applying a substance (e.g., glue, gel, chemical, etc.) to the porous membrane 109, activating a substance on the porous membrane 109, pinching the porous membrane 109, etc. In this manner, the product (e.g., hydrogen peroxide) will remain at the corresponding test and/or control line to react with the glucose and/or metal of the electrodes 202a-f. Although the example of FIG. 2A includes two membrane cuts 201 (e.g., before the first test line/control line and after the last test line/control line), there may be membrane cuts between the different test/control lines and/or the membrane cut before the first test line/control line may be removed. In some examples, the porous membrane 109 can be cut and/or compressed by moving a slider from a first position to a second position, as further described below in conjunction with FIGS. 2K-2Q.

Additionally or alternatively, a mechanical device can apply compression on the porous media 109 to create barriers (e.g., along the lines shown at the membrane cuts 201) that act as a barrier to stop the flow of the product (e.g., hydrogen peroxide) from flowing toward the wicking pad 112. In some examples, the porous membrane 109 can be compressed by moving button from a first position to a second position, as further described below in conjunction with FIGS. 2G-2J. Additionally or alternatively, a chemical substance (e.g., a glue, a gel, an oil, etc.) may be added on or included on and/or in the porous membrane (e.g., along the lines shown at the membrane cuts 201) that acts as a barrier to stop the flow of the product toward the wicking pad 112. Additionally or alternatively, any mechanism (e.g., mechanical, electrical, chemical, etc.) may be used to arrest flow to prevent the product (e.g., hydrogen peroxide) from flowing away from the test zone and/or control zone.

Also, as disclosed herein, in alternative examples, an amperometric redox-reaction without enzymes can be used for bioelectrochemical cell 111a-d. For example, an amperometric signal of the LFA device 104 can be measured without the GOx enzyme because the gold of the AuNP may act as a catalyzer. In such examples, thiosulfate may be used to improve the signal as shown above in Processes 6-8. Additionally, as shown in the above Process 8, thiosulfate, ferricyanide, and KBr or KCl react and the AuNP will catalyze reduction to the ferrocyanide to create an electronic signal.

For example, in FIG. 2A, the electrode 202c is in contact with the T1 test zone and the electrode 202d is in contact with the non-test zone (e.g., the porous media outside of the PC zone, the T1 zone, and the NC zone). In such an example, if the target analyte corresponding to T1 is present in a sample, when the sample is applied to the sample pad 106, the sample will flow into the conjugate pad 108 and bind with conjugates and/or probes labelled with GOx, gold nanoparticles, and/or other label(s) and continue to flow through the porous media 109 and into the wicking pad 112. Because the target analyte is present in the sample, the target analyte attached to the conjugate/probe labelled with the GOx/gold nanoparticles will be immobilized at the test zone T1. Accordingly, after the porous media 109 is saturated with the liquid buffer assay (e.g., a threshold time after the liquid buffer assay including the redox species is applied to the sample pad 106), the glucose is released, and the electrodes 202a-202f are positioned to be in contact with the porous media 109, the GOx/gold nanoparticles at the test zone T1 will oxidize the glucose at the test zone T1 to generate a voltage drop (illustrated as U₂ in FIG. 2A) and/or current between the test zone T1 and the area next to the test zone where the electrode 202d is located. The example wireless chip 114 measures the voltage drop and/or current from the electrode 202c to the electrode 202d and determines that the target analyte is present when the voltage drop and/or current is above a threshold.

In operation, when the example of FIG. 2A is implemented in the LFA device 104 of FIG. 1A, a user or a technician applies a sample (e.g., 25 microliters of urine, blood, etc.) to the example sample pad 106. After the application of the sample, the user or a technician applies the biofuel based buffer (e.g., 6 drops of the liquid assay buffer with a redox agent) to the sample pad 106 and/or a dedicated buffer pad. Once applied, the user waits a threshold amount of time (e.g., 15 minutes) needed for the fuel cell of the porous media 109 to activate. In other examples, the threshold amount of time may be 20 minutes or other values. In some examples, the threshold amount of time is tracked using a software/application-based timer or a timer that is powered by the voltage and/or current generated on the porous media 109. After the threshold duration of time, the user and/or the technician uses the reader 116 to interact with the LFA device to obtain the diagnostic test result (e.g., by generating an electromagnetic field) and within a duration of time (e.g., 1 minute) the wireless chip 114 of the LFA device 104 transmits the results to the reader 116 using the antenna 115. In some examples, the duration of time within which to read the diagnostic test result may be other value such as, for example, less than a minute or more than one or more minutes (e.g., 2 minutes, 3 minutes, or more). In some examples, the reader 116 instructs the user to take one or more actions to cause the glucose to be released from the holding component and the electrodes 202a-202f to come into contact with the porous media 109 and/or arrest flow on the LFA device 104. In such examples, the reader 116 may instruct the user to take the action after within a second duration of time (e.g., 5 minutes) after the threshold amount of time (e.g., the 15 minutes). In some examples, the reader 116 may instruct the user to scan the LFA device 104 to confirm that the correct LFA device has been selected to shear prior to the second duration of time (e.g., the 5 minutes). The amount of time to that the reader 116 needs to power the wireless chip 114 to measure the electrical signal and transmit the measurement signal may be 1-3 seconds. In some examples, the reader 116 instructs the user to confirm that the action to move the electrodes 202a-202f into place and/or arrest flow has been taken. Thereafter, the reader 116 starts a clock, timer, and/or countdown of the duration of time (e.g., the 1 minute) in which the diagnostic test is to be read. In some examples, the reader 116 provides a presentation on the user interface 120 indicative of the times and durations disclosed herein.

FIG. 2B illustrates a side view of the porous media 109 of the machine readable lateral flow immunoassay of FIG. 2A. In the example of FIG. 2B, the layer with the electrodes 202a-f is in direct contact with the porous media 109. In some examples, the electrodes are incorporated in a non-conductive layer, and the non-conductive layer including the electrodes 202a-f is placed (e.g., during manufacturing) in contact with the porous media so that the electrodes 202a-f align as shown in FIG. 2A. In some examples, the electrodes 202a-f are each placed (e.g., during manufacturing) in contact with the porous media 109 to align as shown in FIG. 2A. In some examples, the electrodes 202a-f are not in contact with the porous membrane 109 until when the device is ready to be read. In such examples, a user or device/component can interface and/or engage with a mechanical device (e.g., the push button 228 of FIGS. 2G-2J) to cause the electrodes to come into contact with the porous membrane 109, as further described below.

FIG. 2C illustrates example conjugates that may attach to an immobilized antigen, analyte, and/or antibody on a test zone on and/or coupled to the porous media 109 of the machine readable lateral flow immunoassay of FIGS. 1A-2B for an example HIV-test. FIG. 2C illustrates an example implementation 204. FIG. 2C further includes an HIV-1 target analyte 206, recombinant HIV-1 capture antigens 208, an HIV-2 capture antigen 210, an HIV-1 antibody, an HIV-1 antigen and glucose oxidase complex 211, recombinant immobilized HIV-1 antigens 212, recombinant immobilized HIV-2 antigens 214, and an antiserum 216. Although the example of FIG. 2C corresponds to HIV, the example implementation 204 can be used to detect any target analyte.

During a test, a user and/or patient applies the patient biological sample and/or the buffer solution via the sample pad 106. In the example of FIG. 2C, the sample includes the example HIV-1 target analytes 206. The biological sample and/or buffer flows toward the example wicking pad 112 of FIGS. 1A and 1B. The example conjugate pad 108 is pretreated with the example recombinant HIV-1 capture antigens 208 (e.g., gp41 and p24) and the example HIV-2 capture antigens 210 (e.g., gp 36), which are linked to (e.g., attached to, tagged with, etc.) an enzyme (e.g., glucose oxidase). Because the biological sample includes the example target analyte 206 in FIG. 2C, the example target analyte 206 attaches to the example HIV-1 capture antigen 208 and glucose (e.g., corresponding to a formation of the example HIV-1 antibody, HIV-1 antigen and glucose oxidase complex 211), which is transported toward the wicking pad 112. The test line 1 is coated with the example recombinant immobilized HIV-1 antigens 212 (e.g., gp41 and p24), and the test line 2 is coated with the example recombinant immobilized HIV-2 antigens 214 (e.g., gp36). In case of a positive patient sample with HIV-1 antigens 208, the antibody-antigen-glucose oxidase complex 211 binds to the immobilized recombinant HIV-1 antigen 212 on test line 1. However, because the biological sample does not include an HIV-2 target analyte, nothing binds to the test line 2. As described above, the antigen-antibody complex 211, which is linked to an enzyme (e.g., glucose oxidase) produces a product (e.g., hydrogen peroxide) on the test line 1 when the enzyme reacts with the enzyme substrate (e.g., glucose released from the holding component 218, included in a buffer, coated on the electrodes and/or porous media, etc.). In the example of FIG. 2C, the holding component 218 is a blister, a stamp, a sac, a bag, etc. that holds the enzyme substrate until it is broken, ruptured, dissolved, etc. An example of a breaking of the holding component 218 is further described below in conjunction with FIGS. 2P and/or 2Q. The control line of FIG. 2C includes the example antiserum 216 (e.g., goat anti-recHIV antiserum), which binds to the example recombinant HIV-1 antigens 208. However, if the glucose is applied when the flow of the liquid buffer and/or sample is strong, the glucose can react with glucose oxidase to generate hydrogen peroxide that may flow to outside of the test lines and/or control lines. For example, hydrogen peroxide produced at one of the test lines may flow (e.g., bleed) toward other test and/or control lines, thereby leading to background noise (e.g., when the excess hydrogen peroxide reacts with another electrode to create some electron flow) that may lead to a false positive result.

FIGS. 2D-2F illustrate example front end channels 220, 222, 224 that interface between the example bioelectrochemical cells 111a-d of FIGS. 1A-2C and the wireless chip 114 of FIG. 1A. Although, the example front ends 220, 222, 224 may be implemented as a component outside of the wireless chip 114, the front ends 220, 222, 224 may be implemented in the wireless chip 114 and/or as part of another device. If the example wireless chip 114 is implemented in a standalone device (e.g., separate from the LFA 104), the example front ends 220, 222, 224 could be implemented in the LFA 104 and/or in the device that includes the wireless chip 114.

The example front end channel 220 of FIG. 2D acts as an electronic multiplexer, a group of MOSFETs and/or any other electronic circuit to transmit the signal received from each of the bioelectrochemical cells 111a-d to four inputs of the wireless chip 114. The example wireless chip 114 outputs a control signal to the example front end 220 to control which bioelectrochemical cell signal is to be processed at different points in time. Although the wireless chip 114 of FIG. 2D includes four inputs (e.g., very high, high, low, and very low), the wireless chip 114 can include any number of inputs corresponding to any number of levels. The four inputs of the wireless chip 114 compare the magnitude of the obtained signal to a preset threshold to identify the amount of voltage or current measured. For example, if the output of the bioelectrochemical cells 111a-d is a voltage (e.g., for a potentiometric sensor), the first input may compare the magnitude of the obtained voltage to 80 mV, the second input may compare the magnitude of the obtained voltage to 60 mV, the third input may compare the magnitude of the obtained voltage to 40 mV, and the fourth input may compare the magnitude of the obtained voltage to 20 mV. In this manner, if the front end 220 outputs a 70 mV signal from the first bioelectrochemical cell 111a, the wireless chip 114 will determine that the obtained signal is high (e.g., above 60 mV) but not very high (e.g., above 80 mV) based on the comparisons. The wireless chip 114 may transmit the determined output strength (e.g., very high, high, low, or very low) for each measurement to the example LFA reader application 117. In this manner, the results can be further analyzed to identify patterns, identify the accuracy of a test, and/or for device monitoring purposes (e.g., at the reader 116 and/or at another external device, database, server, or datacenter).

The example front end channel 222 of FIG. 2E is an alternative implementation that acts as a multiplexer and/or a demultiplexer to transmit the signal received (e.g., accessed, obtained, etc.) from each of the bioelectrochemical cells 111a-d to a corresponding input of the wireless chip 114. For example, the front end-channel 222 ensures that the first bioelectrochemical cells 111a is output to the first input, the second bioelectrochemical cells 111b is output to the second input, etc. For example, the front-end channel 222 may be implemented with a multiplexer and a demultiplexer, with the select lines of the multiplexer and demultiplexer coupled together to ensure that the first output of the bioelectrochemical cell 111a is transmitted to the first input of the wireless chip 114, the second output of the bioelectrochemical cell 111b is transmitted to the second input of the wireless chip 114, etc. The example wireless chip 114 outputs one or more control signals to the example front end 220 to control which bioelectrochemical cell signal is to be processed at different points in time. In some examples, the front-end channel 222 may include a multiplexer controlled to sample/measure respective ones of the electrodes at different points in time and each measurement is passed to a single comparator or analog-to-digital converter of the wireless chip 114. In this manner, all the results can be obtained using a single component on the wireless chip 114. In some examples, the front end 222 is controlled to take multiple measurements of each test line and multiple measurements of each control line. For example, the front end 222 is controlled to take multiple measurements of the test lines control lines where the wireless chip 114 transmits multiple results to the reader 116 and/or implements statistical preprocessing of the multiple measurements before sending data to the reader 116. Although the wireless chip 114 of FIG. 2F includes four inputs (e.g., for the four bioelectrochemical cells 111a-111d), the wireless chip 114 can include any number of inputs corresponding to any number of cells, controls, and/or electrodes. In the example of FIG. 2E, the inputs of the wireless chip 114 determine the amount of current and/or voltage (e.g., as digital values) corresponding to the first bioelectrochemical cell 111a-111d. The wireless chip 114 may transmit the determined output strength for each measurement to the example LFA reader application 117.

The example front end channel 224 of FIG. 2F is an alternative implementation that corresponds to direct channels between each of the bioelectrochemical cells 111a-d to a corresponding input of the wireless chip 114. For example, the first output of the bioelectrochemical cell 111a is directly coupled to the first input of the wireless chip 114 via a first channel, the second output of the bioelectrochemical cell 111b is directly coupled to the second input of the wireless chip 114 via a second channel, etc. Although the wireless chip 114 of FIG. 2F includes four inputs (e.g., for the four bioelectrochemical cells 111a-111d), the wireless chip 114 can include any number of inputs corresponding to any number of cells, controls, and/or electrodes. In the example of FIG. 2F, the inputs of the wireless chip 114 determine the amount of current and/or voltage (e.g., as digital values) corresponding to the first bioelectrochemical cell 111a-111d. The wireless chip 114 may transmit the determined output strength for each measurement to the example LFA reader application 117.

Although the example wireless chips 114 of FIGS. 2D, 2E, 2F include a comparator to compare the magnitude of the obtained voltage and/or current to a threshold and transmit a result corresponding to the comparison, the example wireless chips 114 may include an analog-to-digital converter to convert the analog voltage and/or current readings to a digital value and transmit the digital value to the example reader 116. In this manner, the example reader 116 can compare the digital value to one or more thresholds to determine the results. In some examples, the wireless chip 114 may obtain multiple voltage and/or current measurements (e.g., voltage samples, current samples, etc.) for each test line and control line, convert the measurements to digital values, and transmit all the digital values to the example reader 116. In some examples, the wireless chip 114 may perform preprocessing of the values (e.g., statistical analysis such as determining mean, median, mode, standard deviation, etc. of multiple measurements) and transmit one or more value(s) representative of the multiple measurements to the reader 116.

FIG. 2G illustrates an example housing 226 for the example LFA device 104 of FIG. 1A. The example housing 226 includes an example push button 228 and an example sample port or deposit space 230 (e.g., also known as a hole, opening, gap, etc.). In the example of FIG. 2G, the push button 228 is in the first position. The push button 228 can move from a first initial position (e.g., a raised and disengaged position) to a second final position (e.g., a lowered and engaged position), as shown in FIG. 2H.

As described above, a user pushing and/or pressing the push button 228 of FIG. 2G from the first position to the second position causes the electrodes 202a-f to come into contact with the porous media 109 when a test is ready to be read. In some examples, the push button 228 includes or is coupled to a blade. Thus, pushing the push button 228 from the first position to the second position causes the blade of the push button 228 to cut, compress, slice, pinch, etc. the porous media 109 to stop and/or otherwise slow the flow of liquid on the porous media 109, as shown in conjunction with the membrane cuts 201 of FIG. 2A.

Additionally or alternatively, to prevent bleeding of the hydrogen peroxide, one or more blisters may be used. In such examples, the blister may include the redox agent and/or the enzyme substrate at particular locations of the porous media 109 to ensure that the generation of hydrogen peroxide only occurs at particular locations when the target analyte is present. The push button 228 may include text and/or images. Additionally, the push button 228 may be textured to create a grip for a user. A textured portion of the push button 228 may be made of the same or different material as the rest of the push button 228.

The deposit space 230 of FIG. 2G is a slot, opening, hole, etc. where a liquid sample and/or buffer may be applied to the porous media 109 via the sample pad 106 (e.g., the sample pad 106 is located directly below the deposit space 230). In some examples, the deposit space 230 includes two or more deposit spaces (e.g., one for the sample, and one for a buffer). In some examples, the deposit space 230 is sized to match the width of the sample pad 106.

FIG. 2H illustrates the example housing 226 for the LFA device 104 of FIG. 1A in a second position. The housing 226 includes the push button 228 of FIG. 2G pushed into the second position. As described above, pushing the push button 228 into the second position places the electrodes 202a-f of FIG. 2A into contact with the porous media 109 and/or cuts, compresses, slices, and/or pinches one or more portions of the example porous media 109. FIG. 2H also illustrates that text and/or images(s) may be included on the housing 226.

FIG. 2I illustrates an exploded view of the example housing 226 of FIGS. 2G and 2H. The example housing 226 of FIG. 2I includes the push button 228 of FIGS. 2G-2H. The example housing 226 of FIG. 2I further includes an example first portion 232 and an example second portion 234. The first and second portions 232, 234 house an example printed circuitry board (PCB) layer 236 and an example LFA strip 238.

The example first portion 232 is an upper housing of the example housing 226. The example first portion 232 includes a structure to aid in the movement of the example push button 228. For example, the push button 228 includes four guide rings to engage with guideposts of the housing 232 to ensure that the push button 228 does not move laterally from its intended positions. Additionally, the push button 228 may include one or more clips to engage with one or more clips of the lower housing 234. In this manner, when the push button 228 is pushed into the second position from the first position, the clips of the push button 228 and the lower housing 234 engage to cause the push button 228 to remain in the second position (e.g., to keep the electrodes 202a-202f in contact with the porous media 109). For example, the guide ring is slidable along the guidepost to prevent tilting of the push button 228 as the push button 228 moves between the first position and the second position. The example PCB layer 236 houses electronics of the example LFA device 104 of FIG. 1A. For example, the PCB layer 236 may include and/or be connected to the bioelectrochemical cells 111a-d, the wireless chip 114, the antenna 115, the electrodes 202a-f, and/or the example front-end channels 220, 222, 224 of FIGS. 1A-2F. The LFA strip 238 includes the sample pad 106, the conjugate pad 108, the porous media 109, test and/or control portions, the wicking pad 112 of the LFA device 104 of FIGS. 1A-2B. The second portion 234 is a lower housing that holds the upper housing 226 and/or the push button 228 to enclose the PCB layer 236 and the LFA strip 238.

FIG. 2J illustrates a top view of a cross section of the housing 226 of FIG. 2I with respect to the A-A line of FIG. 2G. The example housing 226 supports the example PCB layer 236 of FIG. 2I. The example PCB layer 236 includes an example bridge section 239 that links one side of the PCB layer 236 to another side of the PCB layer 236. The bridge section 239 is structured to be distanced or separated from the sample pad 106 of FIGS. 1A and/or 1B and/or deposit space 230 of FIGS. 2G and/or 2H to avoid the liquid sample and/or buffer from flowing on the PCB layer 236 prior to actuation of the button 228. Thus, the bridge section 239 does not interfere with flow across the LFA strip 238. In some examples, the example PCB layer 236 may be made with flame retardant (FR) 4 (FR-4) with an overall thickness of 0.61 millimeters. The PCB layer 236 may be used as a spring as the button 228 is pressed.

FIG. 2K illustrates an example housing 258 for the example LFA device 104 of FIG. 1A. The example housing 258 includes an example slider 260 and an example sample-port or deposit space 230 (e.g. also known as a hole, gap, etc.). In the example of FIG. 2K, the slider 260 is in the first position. The slider 260 can move from a first position (e.g., where the deposit space 230 is exposed) to a second position (e.g., where the deposit space 230 is covered), as shown in FIG. 2L.

As described above, moving the slider 260 of FIG. 2K from the first position to the second position causes the electrodes 202a-f to come into contact with the porous media 109 when a test is ready to be read. In some examples, moving the slider 260 from the first position to the second position compresses, cuts, slices, pinches, etc. the porous media 109 to stop and/or otherwise slow the flow of liquid on the porous media 109, as shown in conjunction with the membrane cuts 201 of FIG. 2A. The slider 260 may include text and/or images. Additionally, the slider 260 may be textured to create a grip for a user. A textured portion of the slider 260 may be made of the same or different material as the rest of the slider 260.

The deposit space 230 of FIG. 2K is a slot, opening, hole, etc. where a liquid sample and/or buffer may be applied to the porous media 109 via the sample pad 106 (e.g., the sample pad 106 is located directly below the deposit space 230). In some examples, the deposit space 230 includes two or more deposit spaces (e.g., one for the sample, and one for a buffer). In some examples, the example deposit space 230 is sized to match the width of the sample pad 106.

FIG. 2L illustrates the example housing 258 for the LFA device 104 of FIG. 1A in a second position. The housing 258 includes the slider 260 of FIG. 2K moved from the first position of FIG. 2K to the second position. As described above, moving the slider 260 into the second position places the electrodes 202a-f of FIG. 2A into contact with the porous media 109 and/or compresses, cuts, slices, and/or pinches one or more portions of the example porous media 109. In the second position, the slider 260 covers the example deposit space 230. FIG. 2L also illustrates that text and/or images(s) may be included on the housing 258 that is revealed when the example slider 260 is in the second position. In some examples, the text includes instructions such as, for example, instructions for obtaining results of the diagnostic test.

FIG. 2M illustrates an exploded view of the example housing 258 of FIGS. 2K and 2L. The example housing 258 of FIG. 2M includes the slider 260 of FIGS. 2K-2L. The example housing 258 of FIG. 2M further includes an example first portion 262 and an example second portion 264. The first and second portions 262, 264 house an example printed circuitry board (PCB) layer 266 and an example LFA strip 268.

The example first portion 262 is an upper housing of the example housing 258. The example first portion 262 includes a structure to aid in the movement of the example slider 260, as further described below in conjunction with FIG. 2O. The example PCB layer 266 houses electronics of the example LFA device 104 of FIG. 1A. For example, the PCB layer 266 may include and/or be connected to the bioelectrochemical cells 111a-d, the wireless chip 114, the antenna 115, the electrodes 202a-f, and/or the example front-end channels 220, 222, 224 of FIGS. 1A-2F. The LFA strip 268 includes the sample pad 106, the conjugate pad 108, the porous media 109, test and/or control portions, the wicking pad 112 of the LFA device 104 of FIGS. 1A-2B. The second portion 264 is a lower housing that holds the first portion 262 of the housing 258 and/or the slider 260 to enclose the PCB layer 266 and the LFA strip 268.

FIG. 2N illustrates an over-the-top view of a cross section with respect to the B-B line of FIG. 2K of the housing 258 of FIG. 2M. The example housing 258 supports the example PCB layer 266 of FIG. 2M. The example PCB layer 266 includes an example bridge section 267 that links one side of the PCB layer 266 to another side of the PCB layer 266. The bridge section 267 is structured to be distanced or separated from the sample pad 106 of FIGS. 1A and/or 1B and/or deposit space 230 of FIGS. 2K and/or 2L to avoid the liquid sample and/or buffer from flowing on the PCB layer 266 prior to actuation of the slider 260. Thus, the bridge section 267 does not interfere with flow across the LFA strip 268.

FIG. 2O illustrates a cross sectional view with respect to the C-C line of FIG. 2K of the example housing 258 of FIGS. 2K and 2L and rotated 180 degrees horizontally with the slider 260 in the first position (the top figure in FIG. 2O) and the second position (the bottom figure in FIG. 2O). The slider 260 includes an example angled edge 269. The first portion 262 includes an example angled ramp or angled fin 270. The angled edge 269 of the slider 260 is slidably engageable with the fin 270 of the first portion 262. The angled edge 269 and the fin 270 are structured reduce the use-force required to complete actuation of the slider 260 from the first position to the second position. For example, the slopes of the angled edge 269 and the fin 270 correspond to a shallow angle, thereby corresponding to angled contact surface between the angled edge 269 and the fin 270 along which a user applies a lower and more gradual force to move the slider 260 from the first position to a second position that would be applied with a steeper contact surface between the angled edge 269 and the fin 270. Application of force along the contact surface as the slider 260 is moved from the first position to the second position drives vertical displacement of the fin 270 and the first portion 262 and causes compression of the porous media 109 (e.g., causing the electrodes 202a-f on the PCB layer 266 to come into contact with the LFA strip 268 to read an electrical signal to determine a test result). Additionally, the example first portion 262 includes example flow arrestors 271. In the first position, the flow arrestors 271 are located above the LFA strip 268. When the slider 260 moves from the first position to the second position, the angled edge 269 pushes down the fin 270 to cause the flow arrestors 271 to pinch, compress, cut, slice, etc. the example LFA strip 268 to stop and/or slow flow of the sample and/or a buffer on the LFA strip 268.

FIG. 2P illustrates operation of a housing of the example LFA device 104 of FIG. 1A to break the example holding component 218 of FIG. 2C to release glucose. The example LFA device 104 of FIG. 2P includes the example sample pad 106 (e.g., exposed via an opening) of FIG. 1A. The example LFA device 104 further includes an example slider 294 and an example safety latch 296.

At time t1, a user applies a sample to the sample pad 106 via the opening. At time t1, the safety latch 296 is in place to prevent movement of the slider 294. At time t2, the user waits for a duration of time (e.g., 15 minutes) to allow the sample to flow across the porous media 109 of FIG. 1A. At time t3, the user removes and/or discards the safety latch 296. Removing the safety latch 296 unlocks the slider 294. In this manner, the user can slide the slider 294 from a first position to a second position. In some examples, removing the safety latch 296 breaks or otherwise causes the holding component 218 to release glucose and/or a redox agent. From time t4 to t5, the user moves the example slider 294 from the first position (at time t4) to the second position (at time t5). In some examples, moving the slider 294 from the first position to the second position causing the holding component 218 to break and/or release the glucose and/or redox agent housed in the holding component 218. In some examples, movement of the slider 294 may additionally push the electrodes 202a-f into the porous media 109 and/or slide and/or press the porous media 109 to stop and/or slow the flow. In some examples, components of the slider 294 of FIG. 2P may be combined with the slider 260 of FIGS. 2K-2O. In some examples, time t3 and time t4 are substantially contemporaneous.

FIG. 2Q illustrates an alternative operation of a housing of the example LFA device 104 of FIG. 1A to break the example holding component 218 of FIG. 2C to release glucose. The example of FIG. 2Q includes one locking mechanism (e.g., safety latch 296) to prevent movement of the slider 294. The example of FIG. 2Q includes two locking mechanisms, which are disclosed below. The example LFA device 104 of FIG. 2Q includes the example sample pad 106 (e.g., exposed via an opening) of FIG. 1A. The example LFA device 104 further includes the example safety latch 296 and an example slider 298.

At time t1, a user applies a sample to the sample pad 106 via the opening. At time t1, the safety latch 296 (e.g., a first locking mechanism) is in place to prevent movement of the slider 298. In the example of FIG. 2Q, the safety latch 296 is located at one end of the slider 298. At time t2, the user waits for a duration of time (e.g., 15 minutes) to allow the sample to flow across the porous media 109 of FIG. 1A. At time t3, the user removes and/or discards the safety latch 296. Removing the safety latch 296 unlocks the slider 298 to slide in a first direction. In this manner, the user can slide the slider 298 from a first position to a second position. In some examples, removing the safety latch 296 causes the holding component 218 to break and/or release the glucose and/or a redox agent on the porous membrane 109. At time t4, the user moves the example slider 298 in the first direction from the first position to the second position. In some examples, time t3 and time t4 are substantially contemporaneous. Sliding the slider 298 from the first position to the second position unlocks the slider 298 (e.g., unlocks a second locking mechanism). The unlocked slider 298 is able to slide in a second direction (e.g., from the second position to a third position). In some examples, moving the slider 298 from the first position to the second position causes the holding component 218 to break and/or release the glucose and/or a redox agent housed in the holding component 218. In some examples, movement of the slider 298 from the first position to the second position may additionally push the electrodes 202a-f into the porous media 109 and/or slide and/or press the porous media 109 to stop and/or slow the flow.

At time t5, the user slides the slider 298 to from the second position to a third position, as shown at time t6. In some examples, moving the slider 298 from the second position to the third position may cause the holding component 218 to break and/or release the glucose and/or redox agent. Additionally, movement of the slider 298 from the second position to the third position may push the electrodes 202a-f into the porous media 109 and/or slide and/or press the porous media 109 to stop and/or slow the flow. In some examples, components of the slider 298 of FIG. 2Q may be combined with the slider 260 of FIGS. 2K-2O.

FIG. 3 is a block diagram of an example implementation of the machine readable LFA generator 102 of FIG. 1A. The example machine readable LFA generator 102 includes an example user interface 300, an example part generator 302, and an example part applicator 304.

The example user interface 300 of FIG. 3 interfaces with a user and/or manufacturer to obtain instructions regarding how to structure the machine readable LFA device 104. For example, the user interface 300 may receive (e.g., access, obtain, etc.) instructions regarding which type of machine readable LFA device to generate, how many test zones (e.g., for a multiplexing LFA design) and/or test types the LFA device 104 is to perform, and/or how many control zones and/or types to include in the LFA device 104.

The example part generator 302 of FIG. 3 generates and/or obtains the parts of the example LFA device 104. For example, the part generator 302 may generate and/or obtain samples pad 106, the conjugate pad 108, the porous media 109, the antibodies, antigens, and/or molecules to apply to the LFA device 104, the wicking pad 112, the wireless chip 114, the antenna 115, the connections, the housing, the bioelectrochemical cells 111a-n, the example housings 226, etc. from storage. To introduce the enzyme substrate (e.g., glucose), the example part generator 302 may generate the example holding component 218 to house the enzyme substrate (e.g., glucose).

The example part applicator 304 of FIG. 3 applies the obtained and/or generated parts to generate the example machine readable LFA device 104 of FIGS. 1A-2Q based on the user and/or manufacturing instructions. For example, the part applicator 304 may apply antigens and/or antibodies attached to GOx, silver nitrate, reducing agent and/or gold to the obtained and/or generated conjugate pad 108. Additionally, the part applicator 304 structures the parts of the machine readable LFA device 104, applies the antigens and/or antibodies corresponding to the number of tests, types of test, number of controls and/or types of controls into the test and/or control zones of the test area 110 on the porous media 109.

While an example manner of implementing the example machine readable LFA generator 102 of FIG. 1A is illustrated in FIG. 3, one or more of the elements, processes and/or devices illustrated in FIG. 3 may be combined, divided, re-arranged, omitted, eliminated and/or implemented in any other way. Further, the example user interface 300, the example part generator 302, the example part applicator 304, and/or, more generally, the example machine readable LFA generator 102 of FIG. 3 may be implemented by hardware, software, firmware and/or any combination of hardware, software and/or firmware. Thus, for example, any of the example user interface 300, the example part generator 302, the example part applicator 304, and/or, more generally, the example machine readable LFA generator 102 of FIG. 3 could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example user interface 300, the example part generator 302, the example part applicator 304, and/or, more generally, the example machine readable LFA generator 102 of FIG. 3 is/are hereby expressly defined to include a non-transitory computer readable storage device or storage disk such as a memory, a digital versatile disk (DVD), a compact disk (CD), a Blu-ray disk, etc. including the software and/or firmware. Further still, the example machine readable LFA generator 102 of FIG. 3 may include one or more elements, processes and/or devices in addition to, or instead of, those illustrated in FIG. 3, and/or may include more than one of any or all of the illustrated elements, processes, and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

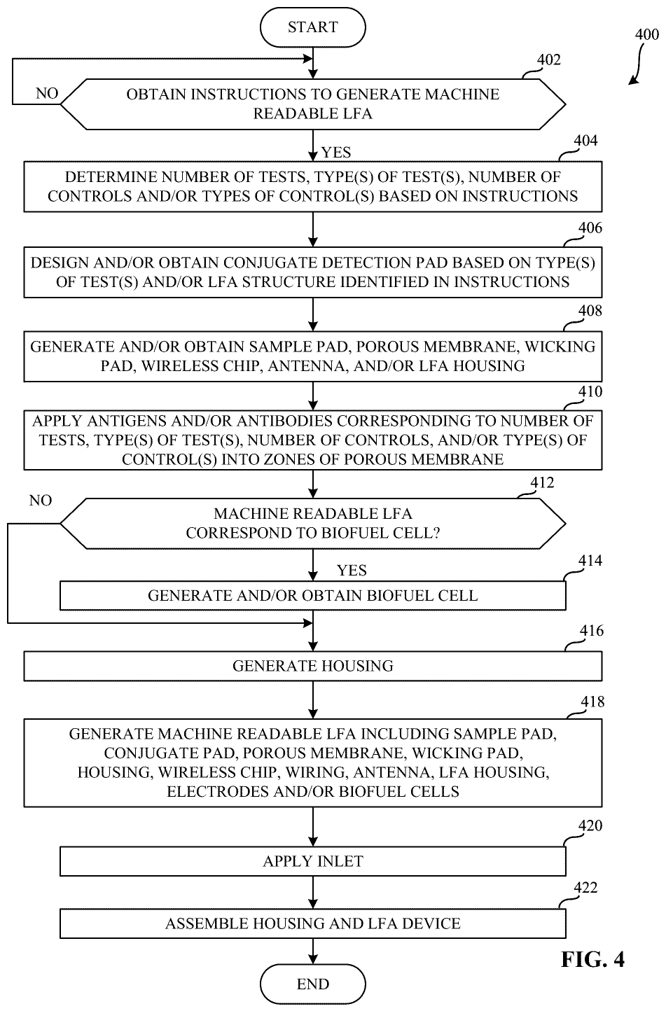

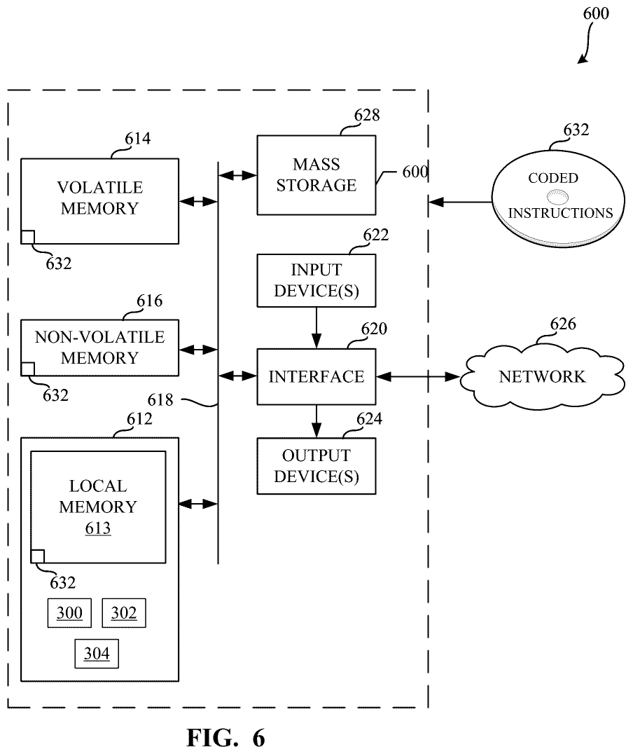

Flowcharts representative of example hardware logic, machine readable instructions, hardware implemented state machines, and/or any combination thereof for implementing the machine readable LFA generator 102 of FIG. 3 are shown in FIGS. 4-5. The machine readable instructions may be one or more executable programs or portion(s) of an executable program for execution by a computer processor such as the processors 612 shown in the example processor platforms 600 discussed below in connection with FIG. 6. The programs may be embodied in software stored on a non-transitory computer readable storage medium such as a CD-ROM, a floppy disk, a hard drive, a DVD, a Blu-ray disk, or a memory associated with the processors 612 but the entire programs and/or parts thereof could alternatively be executed by a device other than the processors 612 and/or embodied in firmware or dedicated hardware. Further, although the example programs are described with reference to the flowcharts illustrated in FIGS. 4-5 many other methods of implementing the example machine readable LFA generator 102 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example processes of FIGS. 5-7 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

FIG. 4 illustrates an example flowchart representative of machine readable instructions 400 that may be executed to implement the example machine readable LFA generator 102 of FIG. 3 to generate one of the example machine readable LFA devices 104 of FIGS. 1A-2Q. Although the instructions 400 of FIG. 4 are described in conjunction with one of the example machine readable LFA device 104 of FIGS. 1A-2Q, the example instructions 400 may be described and/or implemented in conjunction with any type LFA device of any structure.

At block 402, the example user interface 300 determines if instructions were obtained to generate the machine readable LFA device 104. If the example user interface 300 determines that instructions have not been obtained (block 402: NO), control returns to block 402 until instructions are obtained. If the example user interface 300 determines that instructions have been obtained (block 402: YES), the example part generator 302 determines the number of tests, type(s) of test(s), number of control zones, and/or types of control zone(s) based on the user instructions (block 404). For example, the user may provide instructions to generate the LFA device 104 to include three test zones corresponding to three target analytes and one positive control zone.

At block 406, the example part generator 302 designs and/or obtains a conjugate pad based on the type(s) of test(s), and/or the LFA structure identified in the instructions. For example, the part generator 302 may generate (and/or obtain from storage) a conjugate pad including antibodies and/or antigens that attach to target analytes that correspond to the type(s) of test(s) identified in the instructions. For the bioelectrochemical cell LFA device 104, the antigens and/or antibodies are attached to either GOx or gold nanoparticles (e.g., depending on how the bioelectrochemical cells 111a-n are structured).

At block 408, the example part generator 302 generates and/or obtains the example sample pad 106, the example porous media 109, the example wicking pad 112, the example wireless chip 114, the example antenna 115, and/or LFA housing. At block 410, the example part applicator 304 applies (e.g., immobilizes) antigens and/or antibodies corresponding to the target analytes and/or access target analytes to test zones and/or control zones in the test area 110 of the porous media 109. At block 412, the example part generator 302 determines if the instructions correspond to the LFA being the bioelectrochemical cell LFA device 104. If the part generator 302 determines that the LFA does not correspond to the bioelectrochemical cell LFA device 104 (block 412: NO), control continues to block 416. If the part generator 302 determines that the LFA corresponds to the bioelectrochemical cell LFA device 104 (block 412: YES), the example part generator 302 generates and/or obtains a bioelectrochemical cell (block 414). The example part generator 302 generates the bioelectrochemical cell by impregnating a piece of paper or other substance with an enzyme substrate (e.g., glucose) and/or a reducing agent (e.g., hydroquinone, aminophenol, vitamin C, other ascorbic acids, etc.), and/or an electron mediator or a redox-species (e.g. potassium ferricyanide, ferrocene, a ferrocene derivative, etc.). Once impregnated, the example part generator 302 lets the bioelectrochemical cell dry before applying to the LFA device 104.

At block 416, the example part generator 302 generates the example housing 226 to house the lateral flow assay components. As described above, the housing 226 includes components to facilitate the application of a sample onto the porous media 109 and/or press electrodes into place (e.g., in contact with the porous media 109) so that the lateral flow assay can be read by a reader.

At block 418, the example part applicator 304 generates (e.g., assembles and/or applies) one of the machine readable LFAs 104 including the sample pad 106, the example conjugate pad 108, the example porous media 109, the example wicking pad 112, the example wireless chip 114, the housing 226, the example antenna 115, wiring or etching (e.g., to connect the wireless chip 114 to the test zones and/or cells), the electrodes 202a-f, a switch, a button, a slider, flow arrestor(s), electrode board(s), actuator(s), and/or LFA housing. At block 420, the example part applicator 304 applies the example inlet 113 (e.g., by cutting away a portion of the LFA device 104). In some examples, an inlet may not be included. At block 422, the example part applicator 304 assembles the housing 226 and the LFA device 104 so that the LFA device 104 is housed in the housing 226.

FIG. 5 illustrates an example flowchart representative of machine readable instructions 500 that may be executed to implement the example machine readable LFA generator 102 of FIG. 3 to generate one of the example machine readable LFA devices 104 that includes the holding component 218 of FIG. 2C in conjunction with one or more of the structures of FIGS. 1A-2Q. Although the instructions 500 of FIG. 5 are described in conjunction with one of the example machine readable LFA device 104 of FIGS. 1A-2Q, the example instructions 500 may be described and/or implemented in conjunction with any type LFA device of any structure.

At block 502, the example user interface 300 determines if instructions were obtained to generate the machine readable LFA device 104. If the example user interface 300 determines that instructions have not been obtained (block 502: NO), control returns to block 502 until instructions are obtained. If the example user interface 300 determines that instructions have been obtained (block 502: YES), the example part generator 302 determines the number of tests, type(s) of test(s), number of control zones, and/or types of control zone(s) based on the user instructions (block 504). For example, the user may provide instructions to generate the LFA device 104 to include three test zones corresponding to three target analytes and one positive control zone.

At block 506, the example part generator 302 designs and/or obtains a conjugate pad based on the type(s) of test(s), and/or the LFA structure identified in the instructions. For example, the part generator 302 may generate (and/or obtain from storage) a conjugate pad including antibodies and/or antigens that attach to target analytes that correspond to the type(s) of test(s) identified in the instructions. For the bioelectrochemical cell LFA device 104, the antigens and/or antibodies are attached to either GOx or gold nanoparticles (e.g., depending on how the bioelectrochemical cells 111a-n are structured).

At block 508, the example part generator 302 generates and/or obtains the example sample pad 106, the example porous media 109, the example wicking pad 112, the example wireless chip 114, the example antenna 115, and/or LFA housing. At block 510, the example part applicator 304 applies (e.g., immobilizes) antigens and/or antibodies corresponding to the target analytes and/or access target analytes to test zones and/or control zones in the test area 110 of the porous media 109. At block 512, the example part generator 302 determines if the instructions correspond to the LFA being the bioelectrochemical cell LFA device 104. If the part generator 302 determines that the LFA does not correspond to the bioelectrochemical cell LFA device 104 (block 512: NO), control continues to block 516. If the part generator 302 determines that the LFA corresponds to the bioelectrochemical cell LFA device 104 (block 512: YES), the example part generator 302 generates and/or obtains a bioelectrochemical cell (block 514). The example part generator 302 generates the bioelectrochemical cell by impregnating a piece of paper or other substance with an enzyme substrate (e.g. glucose) and/or a reducing agent (e.g. hydroquinone, aminophenol, vitamin C, other ascorbic acids, etc.), and/or an electron mediator or a redox-species (e.g. potassium ferricyanide, ferrocene, a ferrocene derivative, etc.). Once impregnated, the example part generator 302 lets the bioelectrochemical cell dry before applying to the LFA device 104.

At block 516, the example part generator 302 generates the example holding component 218 of FIG. 2C to house the enzyme substrate (e.g., glucose). As described above, glucose released from the holding component 218 will react with an enzyme (e.g., GOx, if the target analyte is present) to generate a product (e.g., hydrogen peroxide). In this manner, the product can react with the electrode to generate an electron flow that can be measured as current to determine that the target analyte is present in the sample.

At block 518, the example part applicator 304 generates (e.g., assembles and/or applies) one of the machine readable LFAs 104 including the sample pad 106, the example conjugate pad 108, the example porous media 109, the example wicking pad 112, the example wireless chip 114, the holding component 218 housing the enzyme substrate, the example antenna 115, wiring or etching (e.g., to connect the wireless chip 114 to the test zones and/or cells), the electrodes 202a-f, the slider 260, the flow arrestors 271, the electrode board(s), the actuator, and/or LFA housing. At block 520, the example part applicator 304 applies the example inlet 113 (e.g., by cutting away a portion of the LFA device 104). In some examples, an inlet may not be included.

FIG. 6 is a block diagram of an example processor platform 600 structured to execute the instructions of FIGS. 4-5 to implement the machine readable LFA generator 102 of FIG. 3. The processor platform 600 can be, for example, a server, a personal computer, a workstation, a self-learning machine (e.g., a neural network), an Internet appliance, or any other type of computing device.

The processor platform 600 of the illustrated example includes a processor 612. The processor 612 of the illustrated example is hardware. For example, the processor 612 can be implemented by one or more integrated circuits, logic circuits, microprocessors, GPUs, DSPs, or controllers from any desired family or manufacturer. The hardware processor may be a semiconductor based (e.g., silicon based) device. In this example, the processor implements the example user interface 300, the example part generator 302, and the example part applicator 304.

The processor 612 of the illustrated example includes a local memory 613 (e.g., a cache). The processor 612 of the illustrated example is in communication with a main memory including a volatile memory 614 and a non-volatile memory 616 via a bus 618. The volatile memory 614 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 616 may be implemented by flash memory and/or any other desired type of memory device. Access to the main memory 614, 616 is controlled by a memory controller.

The processor platform 600 of the illustrated example also includes an interface circuit 620. The interface circuit 620 may be implemented by any type of interface standard, such as an Ethernet interface, a universal serial bus (USB), a Bluetooth® interface, a near field communication (NFC) interface, and/or a PCI express interface.

In the illustrated example, one or more input devices 622 are connected to the interface circuit 620. The input device(s) 622 permit(s) a user to enter data and/or commands into the processor 612. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 624 are also connected to the interface circuit 620 of the illustrated example. The output devices 624 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 620 of the illustrated example, thus, typically includes a graphics driver card, a graphics driver chip and/or a graphics driver processor.

The interface circuit 620 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 626. The communication can be via, for example, an Ethernet connection, a digital subscriber line (DSL) connection, a telephone line connection, a coaxial cable system, a satellite system, a line-of-site wireless system, a cellular telephone system, etc.

The processor platform 600 of the illustrated example also includes one or more mass storage devices 628 for storing software and/or data. Examples of such mass storage devices 628 include floppy disk drives, hard drive disks, compact disk drives, Blu-ray disk drives, redundant array of independent disks (RAID) systems, and digital versatile disk (DVD) drives.

The machine executable instructions 632 of FIG. 6 may be stored in the mass storage device 628, in the volatile memory 614, in the non-volatile memory 616, and/or on a removable non-transitory computer readable storage medium such as a CD or DVD.

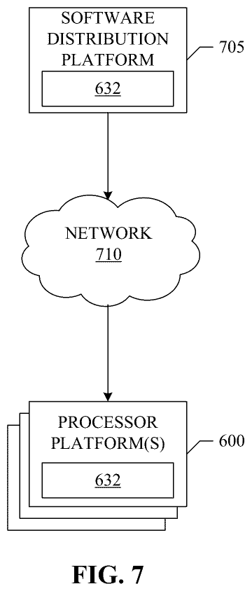

A block diagram illustrating an example software distribution platform 705 to distribute software such as the example computer readable instructions 632 of FIG. 6 to third parties is illustrated in FIG. 7. The example software distribution platform 705 may be implemented by any computer server, data facility, cloud service, etc., capable of storing and transmitting software to other computing devices. The third parties may be customers of the entity owning and/or operating the software distribution platform. For example, the entity that owns and/or operates the software distribution platform may be a developer, a seller, and/or a licensor of software such as the example computer readable instructions 632 of FIG. 6. The third parties may be consumers, users, retailers, OEMs, etc., who purchase and/or license the software for use and/or re-sale and/or sub-licensing. In the illustrated example, the software distribution platform 705 includes one or more servers and one or more storage devices. The storage devices store the computer readable instructions 632 of FIG. 6, which may correspond to the example computer readable instructions 400, 500 of FIGS. 4 and/or 5 as described above. The one or more servers of the example software distribution platform 705 are in communication with a network 710, which may correspond to any one or more of the Internet and/or any of the example networks described above. In some examples, the one or more servers are responsive to requests to transmit the software to a requesting party as part of a commercial transaction. Payment for the delivery, sale and/or license of the software may be handled by the one or more servers of the software distribution platform and/or via a third party payment entity. The servers enable purchasers and/or licensors to download the computer readable instructions 632 from the software distribution platform 705. For example, the software, which may correspond to the example computer readable instructions 400, 500 of FIGS. 4 and/or 5, may be downloaded to the example processor platform 600, which is to execute the computer readable instructions 632 to implement the machine readable LFA generator 102 of FIGS. 1 and/or 3. In some example, one or more servers of the software distribution platform 705 periodically offer, transmit, and/or force updates to the software (e.g., the example computer readable instructions 632 of FIG. 6) to ensure improvements, patches, updates, etc. are distributed and applied to the software at the end user devices.

In addition, as discussed above, visual interpretation of test results is prone to operator subjectivity and error. More accurate and objective results can be obtained with the electrical-based examples disclosed herein. Examples disclosed herein include an LFA device that generates an electrical signal (e.g., a current, a voltage, etc.) using various techniques (e.g., a bioelectrochemical cell technique) that corresponds to a test result. The electrical signal can be measured and wirelessly provided to a reader without a battery device included in the LFA device. In some examples, the LFA device can determine the test result based on the electrical signal and transmit the result to the reader. The reader can obtain the test result and/or process the electrical signal to determine a test result distribute the test result to an external server or database (e.g., via the reader) to provide near real-time data corresponding to disease and/or the spread of a disease.

Further, examples disclosed herein provide a mechanism for arresting flow and/or positioning electrodes on the LFA device to obtain a stronger electrical signal that is able to be measured for a longer duration for time than other diagnostic tests. The examples disclosed herein also enable multiplexing where multiple lines may be used to detect multiple marks (e.g., multiple types of antibodies/antigens) based on one sample that would be difficult or impossible to decern with the human eye. Multiplexing has applications, for example, in a single diagnostic test that could test for multiple types of venereal diseases.

The example controls disclosed herein also control the run time of the diagnostic tests to ensure that an operator does not attempt to obtain results too soon (i.e., before the test is complete), obtain results too late, arrest flow too soon, arrest flow too late, etc. This provides further error proofing in obtaining accurate testing results. Additionally, the example controls facilitate multiple scans to (a) identify the LFA device, (b) verify that the test being read corresponds to the test that is supposed to be read (e.g., when performing multiple tests on different LFAs in parallel using a single reader), and (c) obtain electrical signals corresponding to the test results. This provide further error proofing in obtaining multiple tests with overlapping read windows to ensure that the wrong test is not read.

Examples disclosed herein also include additional information (lot number, expiration date, expiry date, test information, signal quality information, chain of custody, etc.) with the tests results, which can all be transferred together with the NFC technology. Traditional methods use additional readers such as, for example, a bar code reader to read information about a test, which would be in additional to an optical reader that determines/interprets the test results. Thus, the disclosed examples reduce the number of readers needed to obtain a greater amount of information.

Also, the example rapid diagnostic testing using the electronic-based approach have higher sensitivity than conventional, visually read tests. In the examples disclosed herein, a lower detection threshold is possible using the electronic-based technology than can be employed when the results are based on the visual interpretation of an operator.

The examples disclosed herein may include NFC chips that can be encoded and encrypted. The associated tests and manufacturing specific data can subsequently be re-coded if adjustments are made to the product labelling data including, for example, to extend shelf-life and/or if other retrofitting is desired. In some examples, the encoding and/or encryption can be updated and/or configured remotely.

The example controls disclosed herein also apply a potential (e.g., a voltage, a poised potential, etc.) to electrodes in contact with the porous media of a lateral flow assay. Applying the potential and/or voltage aids, facilitates, and/or creates a more ideal environment for a bioelectrochemical reaction to occur. In this manner, applying a potential and/or voltage increases signal robustness and sensitivity. In some examples, rather than using a traditional battery to apply the potential and/or voltage, examples disclosed herein utilize the voltage used to power the wireless chip (e.g., corresponding to the electromagnetic field obtained via an antenna). In this manner, a battery is not required to power the wireless device and/or apply the potential and/or voltage.

Additionally, examples disclosed provide a housing that allows for a sample to be applied to lateral flow assay and allows for the user or autonomous device to push the electrodes into place when the device is ready to be read. The housings may include compartments to hold a buffer and release the buffer with a sample onto a sample pad. In this manner, a user does not need to apply a separate buffer and can facilitate a test by simple tasks (e.g., placing a swab in a compartment, sliding a slider, pressing a button), which reduces the risk of user error.

Example methods, apparatus, systems, and articles of manufacture to make and/or process a diagnostic test device are disclosed herein. Further examples and combinations thereof include the following: Example 1 includes a housing for a lateral flow assay device comprising a first portion including an opening, a second portion coupled to the first portion and house a lateral flow assay strip, the second portion including a first clip, and a push button located within the opening of the first portion, the push button moveable from a first position to a second position, the push button including a second clip to engage the first clip of the second portion to maintain the push button in the second position when moved into the second position.

Example 2 includes the housing of example 1, wherein the push button is to cause electrodes to contact the lateral flow assay strip when the push button moves to the second position.

Example 3 includes the housing of example 1, wherein the push button includes a blade to cause a cut or compression of the lateral flow assay strip when the push button moves to the second position.

Example 4 includes the housing of example 1, wherein the push button includes a guide ring, and the first portion includes a guidepost to engage the guide ring.

Example 5 includes the housing of example 1, wherein the second portion includes a rib to prevent the push button to moving beyond the second position.

Example 6 includes the housing of example 1, wherein the push button is to press a printed circuit board layer in contact with the lateral flow assay strip.

Example 7 includes the housing of example 6, wherein the printed circuit board layer is made with a flame retardant 4 (FR-4) material.

Example 8 includes the housing of example 1, wherein the push button includes a rounded top surface.

Example 9 includes an assay device comprising a first portion, a second portion coupled to the first portion, the second portion including a first clip, a lateral flow assay strip between the first portion and the second portion, and a push button moveable from a first position to a second position between the first portion and the second portion, the push button including a second clip to engage the first clip of the second portion to maintain the push button in the second position when moved into the second position.

Example 10 includes the assay device of example 9, further including a printed circuit board layer, wherein the push button is to press the printed circuit board layer into contact with the lateral flow assay strip when the push button is in the second position.

Example 11 includes the assay device of example 9, wherein the push button includes a guide ring and one of the first portion or the second portion includes a guidepost, the guide ring slidable along the guidepost to prevent tilting of the push button as the push button moves between the first position and the second position.

Example 12 includes a housing for a lateral flow assay device comprising a first portion including an angled fin, a second portion structured to engage the first portion and house a lateral flow assay strip, and a slider structured to move from a first position to a second position by engaging the angled fin of the first portion.

Example 13 includes the housing of example 12, wherein the second portion houses electronics.

Example 14 includes the housing of example 12, wherein the first portion includes a deposit space that is covered by the slider in the second position.

Example 15 includes the housing of example 14, wherein the deposit space is located above a sample pad of the lateral flow assay strip.

Example 16 includes the housing of example 12, wherein the slider is to cause electrodes to contact the lateral flow assay strip when the slider moves to the second position.

Example 17 includes the housing of example 12, wherein the slider is to cause a cut or compression of the lateral flow assay strip when the slider moves to the second position.

Example 18 includes the housing of example 12, wherein the first portion includes a cutout to allow bowing at a center of the first portion.

Example 19 includes the housing of example 12, wherein the slider includes an angled edge to engage the angled fin.

Example 20 includes the housing of example 19, wherein a first slope of the angled edge complements a second slope of the angled fin.

Example 21 includes the housing of example 20, wherein movement of the slider from the first position to the second position causes the angled edge to push the angled fin to cause electrodes to contact the lateral flow assay strip.

Example 22 includes the housing of example 12, further including a printed circuit board layer having a bridge spaced from a sample pad of the lateral flow assay device.

Example 23 includes the housing of example 12, wherein the first portion further includes flow arrestors to at least one of cut or press a portion of the lateral flow assay strip when the slider moves to the second position.

Example 24 includes a lateral flow assay device comprising a conjugate pad including conjugates corresponding to a target analyte in a biological sample, the conjugates labeled with an enzyme, a porous media including a first zone and a second zone, the first zone including at least one of immobilized antigens corresponding to the target analyte or immobilized antibodies corresponding to the target analyte, the second zone laterally displaced from the first zone along a longitudinal axis of the lateral flow assay device, and the porous media to propagate a flow of the biological sample, the conjugates with the enzyme, and a liquid buffer along the porous media, a holding component including an enzyme substrate, a first electrode to contact the porous media in the first zone, and a second electrode to contact the porous media in the second zone, the first and second electrodes to detect an electrical signal when the target analyte is present in the biological sample and the enzyme reacts with the enzyme substrate.

Example 25 includes the lateral flow assay device of example 24, wherein the holding component is to dissolve in response to exposure to at least one of the biological sample or the liquid buffer.

Example 26 includes the lateral flow assay device of example 24, wherein the holding component is to release the enzyme substrate.

Example 27 includes the lateral flow assay device of example 24, wherein the holding component is to release the enzyme substrate after a buffer is applied.

Example 28 includes the lateral flow assay device of example 24, wherein the holding component is to release the enzyme substrate in response to an action taken by a user.

Example 29 includes the lateral flow assay device of example 24, wherein the enzyme is glucose oxidase, and the enzyme substrate is glucose.

Example 30 includes the lateral flow assay device of example 29, wherein the glucose oxidase reacting with the glucose generates hydrogen peroxide, the hydrogen peroxide to react with a metal of the first electrode to generate the electrical signal.

Example 31 includes an apparatus for use with a fluid sample, the apparatus comprising a sample pad, a porous media in contact with the sample pad, a holding component to (a) hold glucose and (b) release the glucose, an electrode, and a mechanical component to move the electrode from a first position to a second position, the electrode to be in contact with the porous media in the second position.

Example 32 includes the apparatus of example 31, wherein the mechanical component is a switch.

Example 33 includes the apparatus of example 31, wherein the mechanical component causes the holding component to release the glucose.

Example 34 includes the apparatus of example 31, wherein the holding component is to dissolve to release the glucose.

Example 35 includes the apparatus of example 31, wherein the porous media includes a zone including at least one of immobilized antigens corresponding to a target analyte or immobilized antibodies corresponding to the target analyte.

Example 36 includes the apparatus of example 35, wherein glucose oxidase attaches to the zone when the target analyte is present in a sample, the released glucose to react with the glucose oxidase to generate hydrogen peroxide, the hydrogen peroxide to react with a metal of the electrode to generate an electrical signal.

Example 37 includes a non-visual indicating biosensor comprising a conjugate pad including conjugates to attach to a target analyte in a biological sample, the conjugates labeled with an enzyme, a porous media having a first end and a second end, the conjugate pad coupled to the first end of the porous media, at least one of immobilized antigens corresponding to the target analyte or immobilized antibodies corresponding to the target analyte coupled to porous media between the first end and the second end, a holding component to release an enzyme substrate, the porous media implementing a bioelectrochemical cell between the first end and the second end downstream from the at least one of immobilized antigens or immobilized antibodies, the bioelectrochemical cell to generate an electrical signal based on a reaction between the released enzyme substrate and the enzyme when the target analyte is present in the biological sample, and a wicking pad to draw fluid along the porous media.

Example 38 includes the non-visual indicating biosensor of example 37, further including an electrode, and a switch to move the electrode into a first position and a second position.

Example 39 includes the non-visual indicating biosensor of example 38, wherein the switch causes the holding component to release the enzyme substrate when placed in the second position.

Example 40 includes the non-visual indicating biosensor of example 37, wherein the holding component is to dissolve to release the enzyme substrate.

Example 41 includes the non-visual indicating biosensor of example 37, wherein the enzyme is glucose oxidase, and the enzyme substrate is glucose.

Example 42 includes the non-visual indicating biosensor of example 41, wherein the glucose oxidase reacting with the glucose generates hydrogen peroxide, the hydrogen peroxide to react with a metal of an electrode to generate the electrical signal.

Example 43 includes a lateral flow assay comprising a porous media, and a holding component including an enzyme substrate, the enzyme substrate to react with an enzyme immobilized on the porous media to generate a product when a target analyte is present in a sample applied to the porous media, and an electrode to react with the product to generate an electrical signal.

Example 44 includes the lateral flow assay of example 43, wherein the holding component is to release the enzyme substrate when ruptured or dissolved.

Example 45 includes the lateral flow assay of example 43, wherein the enzyme is glucose oxidase, the enzyme substrate is glucose, and the product is hydrogen peroxide.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

The following claims are hereby incorporated into this Detailed Description by this reference, with each claim standing on its own as a separate embodiment of the present disclosure.

1. A housing for a lateral flow assay device comprising:
a first portion including an opening;
a second portion coupled to the first portion and house a lateral flow assay strip, the second portion including a first clip; and
a push button located within the opening of the first portion, the push button moveable from a first position to a second position, the push button including a second clip to engage the first clip of the second portion to maintain the push button in the second position when moved into the second position.

2. The housing of claim 1, wherein the push button is to cause electrodes to contact the lateral flow assay strip when the push button moves to the second position.

3. The housing of claim 1, wherein the push button includes a blade to cause a cut or compression of the lateral flow assay strip when the push button moves to the second position.

4. The housing of claim 1, wherein:
the push button includes a guide ring; and
the first portion includes a guidepost to engage the guide ring.

5. The housing of claim 1, wherein the second portion includes a rib to prevent the push button to moving beyond the second position.

6. The housing of claim 1, wherein the push button is to press a printed circuit board layer in contact with the lateral flow assay strip.

7. The housing of claim 6, wherein the printed circuit board layer is made with a flame retardant 4 (FR-4) material.

8. The housing of claim 1, wherein the push button includes a rounded top surface.

9. An assay device comprising:
a first portion;
a second portion coupled to the first portion, the second portion including a first clip;
a lateral flow assay strip between the first portion and the second portion; and
a push button moveable from a first position to a second position between the first portion and the second portion, the push button including a second clip to engage the first clip of the second portion to maintain the push button in the second position when moved into the second position.

10. The assay device of claim 9, further including a printed circuit board layer, wherein the push button is to press the printed circuit board layer into contact with the lateral flow assay strip when the push button is in the second position.

11. The assay device of claim 9, wherein the push button includes a guide ring and one of the first portion or the second portion includes a guidepost, the guide ring slidable along the guidepost to prevent tilting of the push button as the push button moves between the first position and the second position.

12. A housing for a lateral flow assay device comprising:
a first portion including an angled fin;
a second portion structured to engage the first portion and house a lateral flow assay strip; and
a slider structured to move from a first position to a second position by engaging the angled fin of the first portion.

13. The housing of claim 12, wherein the second portion houses electronics.

14. The housing of claim 12, wherein the first portion includes a deposit space that is covered by the slider in the second position.

15. The housing of claim 14, wherein the deposit space is located above a sample pad of the lateral flow assay strip.

16. The housing of claim 12, wherein the slider is to cause electrodes to contact the lateral flow assay strip when the slider moves to the second position.

17. The housing of claim 12, wherein the slider is to cause a cut or compression of the lateral flow assay strip when the slider moves to the second position.

18. The housing of claim 12, wherein the first portion includes a cutout to allow bowing at a center of the first portion.

19. The housing of claim 12, wherein the slider includes an angled edge to engage the angled fin.

20. The housing of claim 19, wherein a first slope of the angled edge complements a second slope of the angled fin.

21.-45. (canceled)
